SEQUENCE LISTING (1 sequence)

SEQ ID NO: 1 - protein, 15 aa, Artificial Sequence
  Completely Synthetic Amino Acid Sequence
EQEDEPEGDYFEWLE

(19) United States
(12) Patent Application Publication
(10) Pub. No.: US 2017/0362243 A1
(43) Pub. Date: Dec. 21, 2017

(54) BIARYLETHER IMIDAZOPYRAZINE BTK INHIBITORS

(71) Applicants: JIAN LIU, EDISON, NJ (US); JOSEPH A. KOZLOWSKI, PRINCETON, NJ (US); SOBHANA BABU BOGA, SCOTCH PLAINS, NJ (US); XIAOLEI GAO, BRIDGEWATER, NJ (US); DEODIAL GUY GUIADEEN, CHESTERFIELD, NJ (US); JYHSHING WANG, WESTFIELD, NJ (US); SHILAN LIU, SHANGHAI (CN)

(72) Inventors: JIAN LIU, EDISON, NJ (US); JOSEPH A. KOZLOWSKI, PRINCETON, NJ (US); SOBHANA BABU BOGA, SCOTCH PLAINS, NJ (US); XIAOLEI GAO, BRIDGEWATER, NJ (US); DEODIAL GUY GUIADEEN, CHESTERFIELD, NJ (US); JYHSHING WANG, WESTFIELD, NJ (US); SHILAN LIU, SHANGHAI (CN)

(73) Assignee: MERCK SHARP & DOHME CORP., RAHWAY, NJ (US)

(21) Appl. No.: 15/538,925

(22) PCT Filed: Dec. 17, 2015
(86) PCT No.: PCT/US15/66223

§ 371 Date: Jun. 22, 2017

(30) Foreign Application Priority Data
Dec. 31, 2014 (CN) PCT/CN2014/095826

(51) Int. Cl. C07D 487/04; A61K 31/4985

(52) U.S. Cl. CPC C07D 487/04; A61K 31/4985

(57) ABSTRACT

The present invention provides Bruton's Tyrosine Kinase (Btk) inhibitor compounds according to Formula (I), or pharmaceutically acceptable salts thereof, or to pharmaceutical compositions comprising these compounds and to their use in therapy. In particular, the present invention relates to the use of Btk inhibitor compounds of Formula (I) in the treatment of Btk mediated disorders.

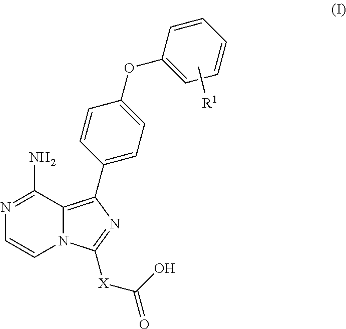

### FIELD OF THE INVENTION

The present invention relates to Btk inhibitor compounds, to pharmaceutical compositions comprising these compounds and to their use in therapy. In particular, the present invention relates to the use of Btk inhibitor compounds in the treatment of Bruton's Tyrosine Kinase (Btk) mediated disorders.

### BACKGROUND OF THE INVENTION

B lymphocyte activation is key in the generation of adaptive immune responses. Derailed B lymphocyte activation is a hallmark of many autoimmune diseases and modulation of this immune response is therefore of therapeutic interest. Recently the success of B cell therapies in autoimmune diseases has been established. Treatment of rheumatoid arthritis (RA) patients with Rituximab (anti-CD20 therapy) is an accepted clinical therapy by now. More recent clinical trial studies show that treatment with Rituximab also ameliorates disease symptoms in relapsing remitting multiple sclerosis (RRMS) and systemic lupus erythematosus (SLE) patients. This success supports the potential for future therapies in autoimmune diseases targeting B cell immunity.

Bruton tyrosine kinase (Btk) is a Tec family non-receptor protein kinase, expressed in B cells and myeloid cells. The function of Btk in signaling pathways activated by the engagement of the B cell receptor (BCR) and FcεR1 on mast cells is well established. In addition, a function for Btk as a downstream target in Toll-like receptor signaling was suggested. Functional mutations in Btk in human results in the primary immunodeficiency disease called XLA which is characterized by a defect in B cell development with a block between pro- and pre-B cell stage. This results in an almost complete absence of B lymphocytes in human causing a pronounced reduction of serum immunoglobulin of all classes. These finding support the key role for Btk in the regulation of the production of auto-antibodies in autoimmune diseases. In addition, regulation of Btk may affect BCR-induced production of pro-inflammatory cytokines and chemokines by B cells, indicating a broad potential for Btk in the treatment of autoimmune diseases.

With the regulatory role reported for Btk in FcεR-mediated mast cell activation, Btk inhibitors may also show potential in the treatment of allergic responses [Gilfillan et al, Immunological Reviews 288 (2009) pp 149-169].

Furthermore, Btk is also reported to be implicated in RANKL-induced osteoclast differentiation [Shinohara et al, Cell 132 (2008) pp 794-806] and therefore may also be of interest for the treatment of bone resorption disorders.

Other diseases with an important role for dysfunctional B cells are B cell malignancies. Indeed anti-CD20 therapy is used effectively in the clinic for the treatment of follicular lymphoma, diffuse large B-cell lymphoma and chronic lymphocytic leukemia [Lim et al, Haematologica, 95 (2010) pp 135-143]. The reported role for Btk in the regulation of proliferation and apoptosis of B cells indicates there is potential for Btk inhibitors in the treatment of B cell lymphomas as well. Inhibition of Btk seems to be relevant in particular for B cell lymphomas due to chronic active BCR signaling [Davis et al, Nature, 463 (2010) pp 88-94].

Some classes of Btk inhibitor compounds have been described as kinase inhibitors, e.g. Imidazo[1,5-f][1,2,4]triazine compounds have been described in WO2005097800 and WO2007064993. Imidazo[1,5-a]pyrazine compounds have been described in WO2005037836 and WO2001019828 as IGF-1R enzyme inhibitors.

Some of the Btk inhibitors reported are not selective over Src-family kinases. With dramatic adverse effects reported for knockouts of Src-family kinases, especially for double and triple knockouts, this is seen as prohibitive for the development of Btk inhibitors that are not selective over the Src-family kinases.

Both Lyn-deficient and Fyn-deficient mice exhibit autoimmunity mimicking the phenotype of human lupus nephritis. In addition, Fyn-deficient mice also show pronounced neurological defects. Lyn knockout mice also show an allergic-like phenotype, indicating Lyn as a broad negative regulator of the IgE-mediated allergic response by controlling mast cell responsiveness and allergy-associated traits [Odom et al, J. Exp. Med., 199 (2004) pp 1491-1502]. Furthermore, aged Lyn knock-out mice develop severe splenomegaly (myeloid expansion) and disseminated monocyte/macrophage tumors [Harder et al, Immunity, 15 (2001) pp 603-615]. These observations are in line with hyperresponsive B cells, mast cells and myeloid cells, and increased Ig levels observed in Lyn-deficient mice. Female Src knockout mice are infertile due to reduced follicle development and ovulation [Roby et al, Endocrine, 26 (2005) pp 169-176]. The double knockouts Src^{-/-}Fyn^{-/-} and Src^{-/-}Yes^{-/-} show a severe phenotype with effects on movement and breathing. The triple knockouts Src^{-/-}Fyn^{-/-}Yes^{-/-} die at day 9.5 [Klinghoffer et al, EMBO J., 18 (1999) pp 2459-2471]. For the double knockout Src^{-/-}Hck^{-/-}, two thirds of the mice die at birth, with surviving mice developing osteopetrosis, extramedullary hematopoiseis, anemia, leukopenia [Lowell et al, Blood, 87 (1996) pp 1780-1792].

Hence, an inhibitor that inhibits multiple or all kinases of the Src-family kinases simultaneously may cause serious adverse effects.

### SUMMARY OF THE INVENTION

The present invention provides compounds which inhibit Btk activity, their use for treatment of Btk mediated diseases and disorders, in particular autoimmune diseases and inflammatory diseases, as well as pharmaceutical compositions comprising such compounds and pharmaceutical carriers.

### DETAILED DESCRIPTION

### Definitions

The terms used herein have their ordinary meaning and the meaning of such terms is independent at each occurrence thereof. That notwithstanding, and except where stated otherwise, the following definitions apply throughout the specification and claims. Chemical names, common names, and chemical structures may be used interchangeably to describe the same structure. These definitions apply regardless of whether a term is used by itself or in combination with other terms, unless otherwise indicated. Hence, the definition of “alkyl” applies to “alkyl” as well as the “alkyl” portions of “hydroxyalkyl,” “fluoroalkyl,” “alkoxy”, “alkylene”, etc.

As used herein, and throughout this disclosure, the following terms, unless otherwise indicated, shall be understood to have the following meanings:

The term “alkyl,” as used herein, refers to an aliphatic hydrocarbon group having one of its hydrogen atoms replaced with a bond having the specified number of carbon atoms. In different embodiments, an alkyl group contains, for example, from 1 to 6 carbon atoms (1-6C)alkyl or from 1 to 3 carbon atoms (1-3C)alkyl. In one embodiment, an alkyl group is linear. In another embodiment, an alkyl group is branched. Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl.

Unless otherwise specifically noted as only “unsubstituted” or only “substituted”, alkyl groups are unsubstituted or substituted with 1 to 3 substituents on each carbon atom.

The term “amount effective” or “effective amount” as used herein, refers to an amount of the compound of Formula I and/or an additional therapeutic agent, or a composition thereof, that is effective in producing the desired therapeutic, ameliorative, inhibitory or preventative effect when administered to a subject suffering from a BTK-mediated disease or disorder. In the combination therapies of the present invention, an effective amount can refer to each individual agent or to the combination as a whole, wherein the amounts of all agents administered are together effective, but wherein the component agent of the combination may not be present individually in an effective amount.

The term “halogen”, as used herein, refers to fluorine, chlorine, bromine or iodine. Fluorine, chlorine or bromine being preferred halogens; fluorine being more preferred.

The term “cycloalkyl,” as used herein, refers to a saturated mono- or multicyclic ring system containing up to 10 ring carbon atoms, and no heteroatom. In a like manner the term “(C₃₋₆) cycloalkyl” or (3-6C)cycloalkyl” refers to a saturated ring having from 3 to 6 ring carbon atoms. Non-limiting examples of monocyclic cycloalkyls include cyclopropyl, cyclobutyl, cyclopentyl, and cyclohexyl. In one embodiment, the cycloalkyl is cyclopropyl.

The term “cycloalkenyl”, as used herein, refers to a mono- or multicyclic ring system containing up to 10 ring carbon atoms, and no heteroatom, that includes at least one double bond. In a like manner the term “(C₃₋₆) cycloalkenyl” or (3-6C)cycloalkenyl” refers to a saturated ring having from 3 to 6 ring carbon atoms. Non-limiting examples of monocyclic cycloalkenyls include cyclopropenyl, cyclobutenyl, cyclopentenyl, and cyclohexenyl.

The ring systems of the cycloalkyl and cycloalkenyl groups may be composed of multicyclic ring systems such that two or more rings may be joined together to form a bridged ring system (i.e. bridged cycloalkyl or bridged cycloalkenyl groups). Non-limiting examples of bridged cycloalkyl groups include C₈ and C₉ bridged cycloalkyls such as, for example, the following:

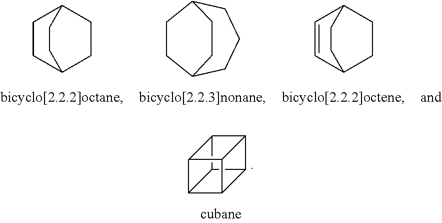 [SMILES: C1CC2CCC1CC2 ; C1CC2CCC(C1)CC2 ; C1=CC2CCC1CC2 ; C12C3C4C1C1C2C3C41]

The term “cycloalkylmethylene”, as used herein, refers to a cycloakyl group as defined above, linked to a methyl group, wherein two of the hydrogen atoms of the methyl group have been replaced with a bond such that the methyl group links the cycloalkyl group to the COOH group of the compound having formula I. The cycloalkyl portion of this substituent may be optionally substituted with one, two or three (1-6C)alkyl groups. The methylene portion of this substituent may be optionally substituted with one or two (1-6C)alkyl groups.

The term “heterocycloalkyl”, as used herein, refers to a heterocycloalkyl group having a 5- or 6-membered saturated ring system having 1 or 2 heteroatoms selected from N and/or O such that the heterocycloalkyl may be linked through a carbon or nitrogen atom. Non-limiting examples of hetercycloalkyls include tetrahydrofuran, tetrahydropyran and piperidine.

The term “C₀” as employed in expressions such as “(C₀₋₆)alkylene” means a direct covalent bond; or when employed in expressions such as “(C₀₋₆)alkyl” means hydrogen. Similarly, when an integer defining the presence of a certain number of atoms in a group is equal to zero, it means that the atoms adjacent thereto are connected directly by a bond; for example, in the structure

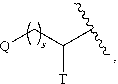

wherein s is an integer equal to zero, 1 or 2, the structure is

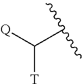

when s is zero; or it means that the indicated atom is absent; for example —S(O)₀— means —S—.

Unless expressly stated to the contrary, all ranges cited herein are inclusive. For example, a heterocycloalkyl described as containing from “1 to 4 heteroatoms” means the heterocycloalkyl can contain 1, 2, 3 or 4 heteroatoms.

When any variable occurs more than one time in any constituent or in any formula depicting and describing compounds of the invention, its definition on each occurrence is independent of its definition at every other occurrence. Also, combinations of substituents and/or variables are permissible only if such combinations result in stable compounds.

For variable definitions containing terms having repeated terms, e.g., (CRiRj)ᵣ, where r is the integer 2, Ri is a defined variable, and Rj is a defined variable, the value of Ri may differ in each instance in which it occurs, and the value of Rj may differ in each instance in which it occurs. For example, if Ri and Rj are independently selected from the group consisting of methyl, ethyl, propyl and butyl, then (CRiRj)₂ can be

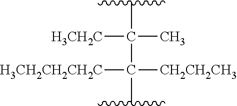

As used herein, the term “Xₐ—X_{b}”, shall have the same meaning as the term “X_{a-b}” or “(a-bX)”, wherein X is any atom and a and b are any integers. For example, “C₁-C₄” shall have the same meaning as “C₁₋₄” or “(1-4C)”. Additionally, when referring to a functional group generically, “A^{x}” shall have the same meaning, and be interchangeable with, “AX”, wherein “A” is any atom and “x” or “X” are any integer. For example, “R¹” shall have the same meaning, and be interchangeable with, “R1”.

In the above definitions with multifunctional groups, the attachment point is at the last group. For example, the term (C₁₋₃)alkoxycarbonyl refers to, e.g.

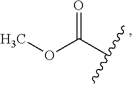 [SMILES: *C(=O)OC]

and the term (C₁₋₄)alkylcarbonyloxy refers to, e.g.

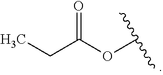 [SMILES: *OC(=O)CC]

The term “purified” as used herein, refers to the physical state of a compound after the compound has been isolated through a synthetic process (e.g., from a reaction mixture), from a natural source, or a combination thereof. The term “purified” also refers to the physical state of a compound after the compound has been obtained from a purification process or processes described herein or well-known to the skilled artisan (e.g., chromatography, recrystallization, and the like), in sufficient purity to be characterizable by standard analytical techniques described herein or well-known to the skilled artisan.

The term “substituted”, as used herein, means that one or more hydrogens on the designated atom/atoms is/are replaced with a selection from the indicated group, provided that the designated atom's normal valency under the existing circumstances is not exceeded, and that the substitution results in a stable compound. Combinations of substituents and/or variables are permissible only if such combinations result in stable compounds. “Stable compound” or “stable structure” is defined as a compound or structure that is sufficiently robust to survive isolation to a useful degree of purity from a reaction mixture, and formulation into an efficacious therapeutic agent.

The term “optionally substituted” means that a compound may or may not be substituted with the specified groups, radicals or moieties.

A “subject” is a human or non-human mammal. In one embodiment, a subject is a human. In another embodiment, the subject is a chimpanzee.

In the above definitions with multifunctional groups, the attachment point is at the last group, unless otherwise specified on the substituent group by a dash. A dash on the substituent group would then represent the point of attachment.

It should be noted that any carbon as well as heteroatom with unsatisfied valences in the text, schemes, examples and tables herein is assumed to have the sufficient number of hydrogen atom(s) to satisfy the valences.

### Compounds of the Invention

The present invention provides Btk inhibitor compounds according to Formula I or pharmaceutically acceptable salts thereof

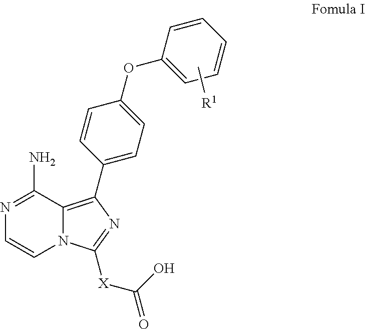

wherein:

R¹ is hydrogen or halogen;

X is selected from the group consisting of:
a) cycloalkyl;
b) cycloalkylmethylene;
c) cycloalkenyl; and
d) heterocycloalkyl;

each optionally substituted with one, two or three (1-6C)alkyl groups.

In one aspect X is selected from the group consisting of:
a) cyclobutyl;
b) cyclopentyl;
c) cyclohexyl;
d) bicyclo[2.2.2]octanyl;
e) bicyclo[2.2.3]nonanyl;
f) bicyclo[2.2.2]octenyl;
g) cubane;
h) tetrahydrofuranyl;
i) tetrahydropyranyl; and
j) piperidinyl.

In another aspect the invention relates to a compound according to Formula I wherein R¹ is halogen. In a preferred aspect, the halogen is fluorine.

The invention also relates to those compounds wherein all specific definitions for R¹ and X, and all substituent groups in the various aspects of the inventions defined hereinabove, occur in any combination within the definition of the Btk inhibitor compounds of Formula I or pharmaceutically acceptable salts thereof.

Non-limiting examples of the compounds of the present invention include:
(3R)-1-[8-amino-1-(4-phenoxyphenyl)imidazo[1,5-a]pyrazin-3-yl]piperidine-3-carboxylic acid;
(1S,3R)-3-{8-amino-1-[4-(3-fluorophenoxy)phenyl]imidazo[1,5-a]pyrazin-3-yl}-1-(1-methylethyl)cyclopentanecarboxylic acid;
(1R,3S)-3-{8-amino-1-[4-(3-fluorophenoxy)phenyl]imidazo[1,5-a]pyrazin-3-yl}-1-(1-methylethyl)cyclopentanecarboxylic acid;
(1R,3R)-3-{8-amino-1-[4-(3-fluorophenoxy)phenyl]imidazo[1,5-a]pyrazin-3-yl}-1-(1-methylethyl)cyclopentanecarboxylic acid;
(1S,3S)-3-{8-amino-1-[4-(3-fluorophenoxy)phenyl]imidazo[1,5-a]pyrazin-3-yl}-1-(1-methylethyl)cyclopentanecarboxylic acid;
4-[8-amino-1-(4-phenoxyphenyl)imidazo[1,5-a]pyrazin-3-yl]bicyclo[2.2.2]octane-1-carboxylic acid;
2-{3-[8-amino-1-(4-phenoxyphenyl)imidazo[1,5-a]pyrazin-3-yl]cyclopentyl}-2-methylpropanoic acid;
(1R,3S)-3-[8-amino-1-(4-phenoxyphenyl)imidazo[1,5-a]pyrazin-3-yl]-1,2,2-trimethylcyclopentanecarboxylic acid;
3-[8-amino-1-(4-phenoxyphenyl)imidazo[1,5-a]pyrazin-3-yl]-methylcyclohexanecarboxylic acid;
(1S,3R)-3-[8-amino-1-(4-phenoxyphenyl)imidazo[1,5-a]pyrazin-3-yl]cyclohexanecarboxylic acid;
cis-4-{8-amino-1-[4-(3-fluorophenoxy)phenyl]imidazo[1,5-a]pyrazin-3-yl}cyclohexanecarboxylic acid;
trans-4-{8-amino-1-[4-(3-fluorophenoxy)phenyl]imidazo[1,5-a]pyrazin-3-yl}cyclohexanecarboxylic acid;
(1S,3R)-3-{8-amino-1-[4-(3-fluorophenoxy)phenyl]imidazo[1,5-a]pyrazin-3-yl}-1-methylcyclohexanecarboxylic acid;
(1 S,3R)-3-[8-amino-1-(4-phenoxyphenyl)imidazo[1,5-a]pyrazin-3-yl]-1-methylcyclohexanecarboxylic acid;
(1R,3S)-3-[8-amino-1-(4-phenoxyphenyl)imidazo[1,5-a]pyrazin-3-yl]-1-methylcyclohexanecarboxylic acid;
(1R,3S)-3-{8-amino-1-[4-(3-fluorophenoxy)phenyl]imidazo[1,5-a]pyrazin-3-yl}-1-methylcyclohexanecarboxylic acid;
trans-4-[8-amino-1-(4-phenoxyphenyl)imidazo[1,5-a]pyrazin-3-yl]cyclohexanecarboxylic acid;
(1S,3R)-3-[8-amino-1-(4-phenoxyphenyl)imidazo[1,5-a]pyrazin-3-yl]-2,2-dimethylcyclobutanecarboxylic acid;
(1 S,3R)-3-[8-amino-1-(4-phenoxyphenyl)imidazo[1,5-a]pyrazin-3-yl]-1-propylcyclohexanecarboxylic acid;
(1R,3R)-3-[8-amino-1-(4-phenoxyphenyl)imidazo[1,5-a]pyrazin-3-yl]-1-propylcyclohexanecarboxylic acid;
5-[8-amino-1-(4-phenoxyphenyl)imidazo[1,5-a]pyrazin-3-yl]bicyclo[3.2.2]nonane-1-carboxylic acid;
4-[8-amino-1-(4-phenoxyphenyl)imidazo[1,5-a]pyrazin-3-yl]bicyclo[2.2.2]oct-2-ene-1-carboxylic acid;
4-(8-amino-1-(4-phenoxyphenyl)imidazo[1,5-a]pyrazin-3-yl)cubane-1-carboxylic acid;
4-[8-amino-1-(4-phenoxyphenyl)imidazo[1,5-a]pyrazin-3-yl]tetrahydro-2H-pyran-4-carboxylic acid;
4-[8-amino-1-(4-phenoxyphenyl)imidazo[1,5-a]pyrazin-3-yl]-1-methylcyclohexanecarboxylic acid;
5-[8-amino-1-(4-phenoxyphenyl)imidazo[1,5-a]pyrazin-3-yl]tetrahydrofuran-2-carboxylic acid;
5-[8-amino-1-(4-phenoxyphenyl)imidazo[1,5-a]pyrazin-3-yl]tetrahydrofuran-2-carboxylic acid;
3-[8-amino-1-(4-phenoxyphenyl)imidazo[1,5-a]pyrazin-3-yl]-1,3-dimethylcyclopentanecarboxylic acid;
3-[8-amino-1-(4-phenoxyphenyl)imidazo[1,5-a]pyrazin-3-yl]-1,3-dimethylcyclopentanecarboxylic acid; and
(1R,3R)-3-[8-amino-1-(4-phenoxyphenyl)imidazo[1,5-a]pyrazin-3-yl]cyclohexanecarboxylic acid.

In another embodiment, the invention provides compounds of Formula Ia or pharmaceutically acceptable salts thereof

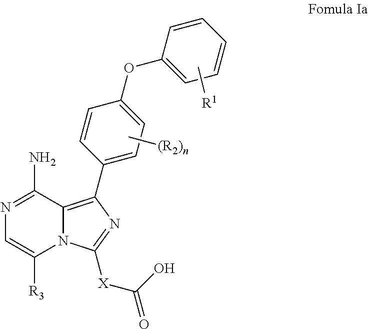

wherein:

n is 0, 1 or 2;

R¹ is hydrogen or halogen;

R₂ is independently selected from the group consisting of methoxy, ethoxy, halogen, and hydroxyl; and

R₃ is hydrogen, halogen, or C(1-3) alkyl; and

X is selected from the group consisting of:
a) cycloalkyl;
b) cycloalkylmethylene;
c) cycloalkenyl; and
d) heterocycloalkyl;

each optionally substituted with one, two or three (1-6C)alkyl groups.

In one aspect X is selected from the group consisting of:
a) cyclobutyl;
b) cyclopentyl;
c) cyclohexyl;
d) bicyclo[2.2.2]octanyl;
e) bicyclo[2.2.3]nonanyl;
f) bicyclo[2.2.2]octenyl;
g) cubane;
h) tetrahydrofuranyl;
i) tetrahydropyranyl; and
j) piperidinyl.

In another aspect the invention relates to a compound according to Formula Ia wherein R¹ is halogen. In a preferred aspect, the halogen is fluorine.

The compounds of this invention include the salts, solvates, hydrates or prodrugs of the compounds. The use of the terms “salt”, “solvate”, “hydrate”, “prodrug” and the like, is intended to equally apply to the salt, solvate, hydrate and prodrug of enantiomers, stereoisomers, rotamers, tautomers, positional isomers, or racemates of the inventive compounds.

### Salts

The Btk inhibitor compounds of the present invention, which can be in the form of a free base, may be isolated from the reaction mixture in the form of a pharmaceutically acceptable salt.

The compounds of Formula I can form salts which are also within the scope of this invention. Reference to a compound of Formula I herein is understood to include reference to pharmaceutically acceptable salts thereof, unless otherwise indicated. The term “pharmaceutically acceptable salt(s)” or “salt”, as employed herein, denotes acidic salts formed with inorganic and/or organic acids, as well as basic salts formed with inorganic and/or organic bases. In addition, when a compound of Formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Such acidic and basic salts used within the scope of the invention are pharmaceutically acceptable (i.e., non-toxic, physiologically acceptable) salts. Salts of the compounds of Formula I may be formed, for example, by reacting a compound of Formula I with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like.

Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

### Crystals

The Btk inhibitor compounds of the present invention may exist as amorphous forms or crystalline forms.

The compounds of Formula I may have the ability to crystallize in more than one form, a characteristic known as polymorphism, and it is understood that such polymorphic forms (“polymorphs”) are within the scope of Formula I. Polymorphism generally can occur as a response to changes in temperature or pressure or both and can also result from variations in the crystallization process. Polymorphs can be distinguished by various physical characteristics known in the art such as x-ray diffraction patterns, solubility and melting point.

### Solvates

The compounds having Formula I or the pharmaceutically acceptable salts may form hydrates or solvates. It is known to those of skill in the art that charged compounds form hydrated species when lyophilized with water, or form solvated species when concentrated in a solution with an appropriate organic solvent. The compounds of this invention include the hydrates or solvates of the compounds listed.

One or more compounds of the invention having Formula I or the pharmaceutically acceptable salts or solvates thereof may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun. 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example IR spectroscopy, show the presence of the solvent (or water) in the crystals as a solvate (or hydrate).

### Optical Isomers

The compounds of Formula I may contain asymmetric or chiral centers, and, therefore, exist in different stereoisomeric forms. It is intended that all stereoisomeric forms of the compounds of Formula I, as well as mixtures thereof, including racemic mixtures, form part of the present invention. In addition, the present invention embraces all geometric and positional isomers. For example, if a compound of Formula I incorporates a double bond or a fused ring, both the cis- and trans-forms, as well as mixtures, are embraced within the scope of the invention. Such stereoisomeric forms also include enantiomers and diastereoisomers, etc.

For chiral compounds, methods for asymmetric synthesis whereby the pure stereoisomers are obtained are well known in the art, e.g. synthesis with chiral induction, synthesis starting from chiral intermediates, enantioselective enzymatic conversions, separation of stereoisomers using chromatography on chiral media. Such methods are described in Chirality in Industry (edited by A. N. Collins, G. N. Sheldrake and J. Crosby, 1992; John Wiley). Likewise methods for synthesis of geometrical isomers are also well known in the art.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g. chiral auxiliary such as a chiral alcohol or Mosher's acid chloride), separating the diastereomers and converting (e.g. hydrolyzing) the individual diastereomers to the corresponding pure enantiomers. Also, some of the compounds of Formula I may be atropisomers (e.g. substituted biaryls) and are considered as part of this invention. Enantiomers can also be separated by use of chiral HPLC column.

It is also possible that the compounds of Formula I may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers. Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

### Prodrugs

A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield a compound of Formula I or a pharmaceutically acceptable salt, hydrate or solvate of the compound. The transformation may occur by various mechanisms (e.g. by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

### Isotopes

In the compounds of Formula I, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I can be prepared without undue experimentation by conventional techniques well known to those skilled in the art or by processes analogous to those described in the Schemes and Examples herein using appropriate isotopically-enriched reagents and/or intermediates.

Certain isotopically-labelled compounds of Formula I (e.g. those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formula I can generally be prepared by following procedures analogous to those disclosed in the Schemes and/or in the Examples herinbelow, by substituting an appropriate isotopically labeled reagent for a non-isotopically labeled reagent.

### Utilities

The compounds having Formula I and pharmaceutical compositions thereof can be used to treat or prevent a variety of conditions, diseases or disorders mediated by Bruton's Tyrosine kinase (Btk). Such Btk-mediated conditions, diseases or disorders include, but are not limited to: (1) arthritis, including rheumatoid arthritis, juvenile arthritis, psoriatic arthritis and osteoarthritis; (2) asthma and other obstructive airways diseases, including chronic asthma, late asthma, airway hyper-responsiveness, bronchitis, bronchial asthma, allergic asthma, intrinsic asthma, extrinsic asthma, dust asthma, adult respiratory distress syndrome, recurrent airway obstruction, and chronic obstruction pulmonary disease including emphysema; (3) autoimmune diseases or disorders, including those designated as single organ or single cell-type autoimmune disorders, for example Hashimoto's thyroiditis, autoimmune hemolytic anemia, autoimmune atrophic gastritis of pernicious anemia, autoimmune encephalomyelitis, autoimmune orchitis, Goodpasture's disease, autoimmune thrombocytopenia including idiopathic thrombopenic purpura, sympathetic ophthalmia, myasthenia gravis, Graves' disease, primary biliary cirrhosis, chronic aggressive hepatitis, ulcerative colitis and membranous glomerulopathy, those designated as involving systemic autoimmune disorder, for example systemic lupus erythematosis, immune thrombocytopenic purpura, rheumatoid arthritis, Sjogren's syndrome, Reiter's syndrome, polymyositis-dermatomyositis, systemic sclerosis, polyarteritis nodosa, multiple sclerosis and bullous pemphigoid, and additional autoimmune diseases, which can be B-cell (humoral) based or T-cell based, including Cogan's syndrome, ankylosing spondylitis, Wegener's granulomatosis, autoimmune alopecia, Type I or juvenile onset diabetes, and thyroiditis; (4) cancers or tumors, including alimentary/gastrointestinal tract cancer, colon cancer, liver cancer, skin cancer including mast cell tumor and squamous cell carcinoma, breast and mammary cancer, ovarian cancer, prostate cancer, lymphoma and leukemia (including but not limited to acute myelogenous leukemia, chronic myelogenous leukemia, mantle cell lymphoma, NHL B cell lymphomas (e.g. precursor B-ALL, marginal zone B cell lymphoma, chronic lymphocytic leukemia, diffuse large B cell lymphoma, Burkitt lymphoma, mediastinal large B-cell lymphoma), Hodgkin lymphoma, NK and T cell lymphomas; TEL-Syk and ITK-Syk fusion driven tumors, myelomas including multiple myeloma, myeloproliferative disorders kidney cancer, lung cancer, muscle cancer, bone cancer, bladder cancer, brain cancer, melanoma including oral and metastatic melanoma, Kaposi's sarcoma, proliferative diabetic retinopathy, and angiogenic-associated disorders including solid tumors, and pancreatic cancer; (5) diabetes, including Type I diabetes and complications from diabetes; (6) eye diseases, disorders or conditions including autoimmune diseases of the eye, keratoconjunctivitis, vernal conjunctivitis, uveitis including uveitis associated with Behcet's disease and lens-induced uveitis, keratitis, herpetic keratitis, conical keratitis, comeal epithelial dystrophy, keratoleukoma, ocular premphigus, Mooren's ulcer, scleritis, Grave's ophthalmopathy, Vogt-Koyanagi-Harada syndrome, keratoconjunctivitis sicca (dry eye), phlyctenule, iridocyclitis, sarcoidosis, endocrine ophthalmopathy, sympathetic ophthalmitis, allergic conjunctivitis, and ocular neovascularization; (7) intestinal inflammations, allergies or conditions including Crohn's disease and/or ulcerative colitis, inflammatory bowel disease, coeliac diseases, proctitis, eosinophilic gastroenteritis, and mastocytosis; (8) neurodegenerative diseases including motor neuron disease, Alzheimer's disease, Parkinson's disease, amyotrophic lateral sclerosis, Huntington's disease, cerebral ischemia, or neurodegenerative disease caused by traumatic injury, strike, glutamate neurotoxicity or hypoxia; ischemic/reperfusion injury in stroke, myocardial ischemica, renal ischemia, heart attacks, cardiac hypertrophy, atherosclerosis and arteriosclerosis, organ hypoxia; (9) platelet aggregation and diseases associated with or caused by platelet activation, such as arteriosclerosis, thrombosis, intimal hyperplasia and restenosis following vascular injury; (10) conditions associated with cardiovascular diseases, including restenosis, acute coronary syndrome, myocardial infarction, unstable angina, refractory angina, occlusive coronary thrombus occurring post-thrombolytic therapy or post-coronary angioplasty, a thrombotically mediated cerebrovascular syndrome, embolic stroke, thrombotic stroke, transient ischemic attacks, venous thrombosis, deep venous thrombosis, pulmonary embolus, coagulopathy, disseminated intravascular coagulation, thrombotic thrombocytopenic purpura, thromboangiitis obliterans, thrombotic disease associated with heparin-induced thrombocytopenia, thrombotic complications associated with extracorporeal circulation, thrombotic complications associated with instrumentation such as cardiac or other intravascular catheterization, intra-aortic balloon pump, coronary stent or cardiac valve, conditions requiring the fitting of prosthetic devices, and the like; (11) skin diseases, conditions or disorders including atopic dermatitis, eczema, psoriasis, scleroderma, pruritus and other pruritic conditions; (12) allergic reactions including anaphylaxis, allergic rhinitis, allergic dermatitis, allergic urticaria, angioedema, allergic asthma, or allergic reaction to insect bites, food, drugs, or pollen; (13) transplant rejection, including pancreas islet transplant rejection, bone marrow transplant rejection, graft-versus-host disease, organ and cell transplant rejection such as bone marrow, cartilage, comea, heart, intervertebral disc, islet, kidney, limb, liver, lung, muscle, myoblast, nerve, pancreas, skin, small intestine, or trachea, and xeno transplantation; and (14) low grade scarring including scleroderma, increased fibrosis, keloids, post-surgical scars, pulmonary fibrosis, vascular spasms, migraine, reperfusion injury, and post-myocardial infarction.

The invention thus provides compounds of Formula I and salts thereof for use in therapy, and particularly in the treatment of disorders, diseases and conditions mediated by inappropriate Btk activity.

The inappropriate Btk activity referred to herein is any Btk activity that deviates from the normal Btk activity expected in a particular mammalian subject. Inappropriate Btk activity may take the form of, for instance, an abnormal increase in activity, or an aberration in the timing and or control of Btk activity. Such inappropriate activity may result then, for example, from overexpression or mutation of the protein kinase leading to inappropriate or uncontrolled activation.

In one embodiment, the present invention provides for the use of a compound of Formula I, or a pharmaceutically acceptable salt thereof for the manufacture of a medicament for the treatment of a Btk-mediated disorder.

In another embodiment, the present invention provides methods of regulating, modulating, or inhibiting Btk for the prevention and/or treatment of disorders related to unregulated or inappropriate Btk activity.

In a further embodiment, the present invention provides a method for treating a subject suffering from a disorder mediated by Btk, which comprises administering to said subject a compound of Formula I or a pharmaceutically acceptable salt thereof in an amount effective to treat the Btk-mediated disorder.

A further aspect of the invention resides in the use of a compound of Formula I or a pharmaceutically acceptable salt thereof for the manufacture of a medicament to be used for the treatment of chronic B cell disorders in which T cells play a prominent role.

Thus, the compounds according to the invention may be used in therapies to treat or prevent Bruton's Tyrosine Kinase (Btk) mediated diseases, conditions and disorders. Btk mediated diseases, conditions and disorders as used herein, mean any disease, condition or disorder in which B cells, mast cells, myeloid cells or osteoclasts play a central role. These diseases include but are not limited to, immune, autoimmune and inflammatory diseases, allergies, infectious diseases, bone resorption disorders and proliferative diseases.

Immune, autoimmune and inflammatory diseases that may be treated or prevented with the compounds of the present invention include rheumatic diseases (e.g. rheumatoid arthritis, psoriatic arthritis, infectious arthritis, progressive chronic arthritis, deforming arthritis, osteoarthritis, traumatic arthritis, gouty arthritis, Reiter's syndrome, polychondritis, acute synovitis and spondylitis), glomerulonephritis (with or without nephrotic syndrome), Goodpasture's syndrome, (and associated glomerulonephritis and pulmonary hemorrhage), atherosclerosis, autoimmune hematologic disorders (e.g. hemolytic anemia, aplasic anemia, idiopathic thrombocytopenia, chronic idiopathic thrombocytopenic purpura (ITP), and neutropenia), autoimmune gastritis, and autoimmune inflammatory bowel diseases (e.g. ulcerative colitis and Crohn's disease), irritable bowel syndrome, host versus graft disease, allograft rejection, chronic thyroiditis, Graves' disease, Sjorgren's disease, scleroderma, diabetes (type I and type II), active hepatitis (acute and chronic), pancreatitis, primary billiary cirrhosis, myasthenia gravis, multiple sclerosis, systemic lupus erythematosis, psoriasis, atopic dermatitis, dermatomyositis, contact dermatitis, eczema, skin sunburns, vasculitis (e.g. Behcet's disease), ANCA-associated and other vasculitudes, chronic renal insufficiency, Stevens-Johnson syndrome, inflammatory pain, idiopathic sprue, cachexia, sarcoidosis, Guillain-Barré syndrome, uveitis, conjunctivitis, kerato conjunctivitis, otitis media, periodontal disease, Addison's disease, Parkinson's disease, Alzheimer's disease, diabetes, septic shock, myasthenia gravis, pulmonary interstitial fibrosis, asthma, bronchitis, rhinitis, sinusitis, pneumoconiosis, pulmonary insufficiency syndrome, pulmonary emphysema, pulmonary fibrosis, silicosis, chronic inflammatory pulmonary disease (e.g. chronic obstructive pulmonary disease) and other inflammatory or obstructive disease on airways.

Allergies that may be treated or prevented include, among others, allergies to foods, food additives, insect poisons, dust mites, pollen, animal materials and contact allergans, type I hypersensitivity allergic asthma, allergic rhinitis, allergic conjunctivitis.

Infectious diseases that may be treated or prevented include, among others, sepsis, septic shock, endotoxic shock, sepsis by Gram-negative bacteria, shigellosis, meningitis, cerebral malaria, pneumonia, tuberculosis, viral myocarditis, viral hepatitis (hepatitis A, hepatitis B and hepatitis C), HIV infection, retinitis caused by cytomegalovirus, influenza, herpes, treatment of infections associated with severe burns, myalgias caused by infections, cachexia secondary to infections, and veterinary viral infections such as lentivirus, caprine arthritic virus, visna-maedi virus, feline immunodeficiency virus, bovine immunodeficiency virus or canine immunodeficiency virus.

Bone resorption disorders that may be treated or prevented include, among others, osteoporosis, osteoarthritis, traumatic arthritis, gouty arthritis and bone disorders related with multiple myeloma.

Proliferative diseases that may be treated or prevented include, among others, non-Hodgkin lymphoma (in particular the subtypes diffuse large B-cell lymphoma (DLBCL) and mantle cell lymphoma (MCL)), B cell chronic lymphocytic leukemia and acute lymphoblastic leukemia (ALL) with mature B cell, ALL in particular.

In particular the compounds of Formula I or pharmaceutically acceptable salts may be used for the treatment of B cell lymphomas resulting from chronic active B cell receptor signaling.

Yet another aspect of the present invention provides a method for treating diseases caused by or associated with Fc receptor signaling cascades, including FceRI and/or FcgRI-mediated degranulation as a therapeutic approach towards the treatment or prevention of diseases characterized by, caused by and/or associated with the release or synthesis of chemical mediators of such Fc receptor signaling cascades or degranulation. In addition, Btk is known to play a critical role in immunotyrosine-based activation motif (ITAM) singaling, B cell receptor signaling, T cell receptor signaling and is an essential component of integrin beta (1), beta (2), and beta (3) signaling in neutrophils. Thus, compounds of the present invention can be used to regulate Fc receptor, ITAM, B cell receptor and integrin signaling cascades, as well as the cellular responses elicited through these signaling cascades. Non-limiting examples of cellular responses that may be regulated or inhibited include respiratory burst, cellular adhesion, cellular degranulation, cell spreading, cell migration, phagocytosis, calcium ion flux, platelet aggregation and cell maturation.

### Combination Therapy

Included herein are methods of treatment and/or pharmaceutical compositions in which at least one compound of Formula I or a pharmaceutically acceptable salt thereof is administered in combination with at least one other active agent. The other active agent is an anti-inflammatory agent, an immunosuppressant agent, or a chemotherapeutic agent. Anti-inflammatory agents include but are not limited to NSAIDs, non-specific and COX-2 specific cyclooxgenase enzyme inhibitors, gold compounds, corticosteroids, methotrexate, tumor necrosis factor receptor (TNF) receptors antagonists, immunosuppressants and methotrexate.

Examples of NSAIDs include, but are not limited to, ibuprofen, flurbiprofen, naproxen and naproxen sodium, diclofenac, combinations of diclofenac sodium and misoprostol, sulindac, oxaprozin, diflunisal, piroxicam, indomethacin, etodolac, fenoprofen calcium, ketoprofen, sodium nabumetone, sulfasalazine, tolmetin sodium, and hydroxychloroquine. Examples of NSAIDs also include COX-2 specific inhibitors such as celecoxib, valdecoxib, lumiracoxib and/or etoricoxib.

In some embodiments, the anti-inflammatory agent is a salicylate. Salicylates include by are not limited to acetylsalicylic acid or aspirin, sodium salicylate, and choline and magnesium salicylates.

The anti-inflammatory agent may also be a corticosteroid. For example, the corticosteroid may be cortisone, dexamethasone, methylprednisolone, prednisolone, prednisolone sodium phosphate, or prednisone.

In additional embodiments the anti-inflammatory agent is a gold compound such as gold sodium thiomalate or auranofin.

The invention also includes embodiments in which the anti-inflammatory agent is a metabolic inhibitor such as a dihydrofolate reductase inhibitor, such as methotrexate or a dihydroorotate dehydrogenase inhibitor, such as leflunomide.

Other embodiments of the invention pertain to combinations in which at least one anti-inflammatory agent is an anti-C5 monoclonal antibody (such as eculizumab or pexelizumab), a TNF antagonist, such as entanercept, or infliximab, which is an anti-TNF alpha monoclonal antibody.

Still other embodiments of the invention pertain to combinations in which at least one active agent is an immunosuppressant agent, such as an immunosuppressant compound chosen from methotrexate, leflunomide, cyclosporine, tacrolimus, azathioprine, and mycophenolate mofetil.

B-cells and B-cell precursors expressing BTK have been implicated in the pathology of B-cell malignancies, including, but not limited to, B-cell lymphoma, lymphoma (including Hodgkin's and non-Hodgkin's lymphoma), hairy cell lymphoma, multiple myeloma, chronic and acute myelogenous leukemia and chronic and acute lymphocytic leukemia.

BTK has been shown to be an inhibitor of the Fas/APO-1 (CD-95) death inducing signaling complex (DISC) in B-lineage lymphoid cells. The fate of leukemia/lymphoma cells may reside in the balance between the opposing proapoptotic effects of caspases activated by DISC and an upstream anti-apoptotic regulatory mechanism involving BTK and/or its substrates (Vassilev et al., J. Biol. Chem. 1998, 274, 1646-1656).

It has also been discovered that BTK inhibitors are useful as chemosensitizing agents, and, thus, are useful in combination with other chemotherapeutic agents, in particular, drugs that induce apoptosis. Examples of other chemotherapeutic agents that can be used in combination with chemosensitizing BTK inhibitors include topoisomerase I inhibitors (camptothecin or topotecan), topoisomerase II inhibitors (e.g. daunomycin and etoposide), alkylating agents (e.g. cyclophosphamide, melphalan and BCNU), tubulin directed agents (e.g. taxol and vinblastine), and biological agents (e.g. antibodies such as anti CD20 antibody, IDEC 8, immunotoxins, and cytokines).

Btk activity has also been associated with some leukemias expressing the bcr-abl fusion gene resulting from translocation of parts of chromosome 9 and 22. This abnormality is commonly observed in chronic myelogenous leukemia. Btk is constitutively phosphorylated by the bcr-abl kinase which initiates downstream survival signals which circumvents apoptosis in bcr-abl cells. (N. Feldhahn et al. J. Exp. Med. 2005 201(11):1837-1852).

The compound(s) of Formula I and the other pharmaceutically active agent(s) may be administered together or separately and, when administered separately this may occur simultaneously or sequentially in any order. The amounts of the compound(s) of Formula I and the other pharmaceutically active agent(s) and the relative timings of administration will be selected in order to achieve the desired combined therapeutic effect.

For the treatment of the inflammatory diseases, rheumatoid arthritis, psoriasis, inflammatory bowel disease, COPD, asthma and allergic rhinitis a compound of Formula I may be combined with one or more other active agents such as: (1) TNF-α inhibitors such as infliximab (Remicade®), etanercept (Enbrel®), adalimumab (Humira®), certolizumab pegol (Cimzia®), and golimumab (Simponi®); (2) non-selective COX-I/COX-2 inhibitors (such as piroxicam, diclofenac, propionic acids such as naproxen, flubiprofen, fenoprofen, ketoprofen and ibuprofen, fenamates such as mefenamic acid, indomethacin, sulindac, etodolac, azapropazone, pyrazolones such as phenylbutazone, salicylates such as aspirin); (3) COX-2 inhibitors (such as meloxicam, celecoxib, rofecoxib, valdecoxib and etoricoxib); (4) other agents for treatment of rheumatoid arthritis including methotrexate, leflunomide, sulfasalazine, azathioprine, cyclosporin, tacrolimus, penicillamine, bucillamine, actarit, mizoribine, lobenzarit, ciclesonide, hydroxychloroquine, d-penicillamine, aurothiomalate, auranofin or parenteral or oral gold, cyclophosphamide, Lymphostat-B, BAFF/APRIL inhibitors and CTLA-4-Ig or mimetics thereof; (5) leukotriene biosynthesis inhibitor, 5-lipoxygenase (5-LO) inhibitor or 5-lipoxygenase activating protein (FLAP) antagonist such as zileuton; (6) LTD4 receptor antagonist such as zafirlukast, montelukast and pranlukast; (7) PDE4 inhibitor such as roflumilast, cilomilast, AWD-12-281 (Elbion), and PD-168787 (Pfizer); (8) antihistaminic H1 receptor antagonists such as cetirizine, levocetirizine, loratadine, desloratadine, fexofenadine, astemizole, azelastine, levocabastine, olopatidine, methapyrilene and chlorpheniramine; (9) α1- and α2-adrenoceptor agonist vasoconstrictor sympathomimetic agent, such as propylhexedrine, phenylephrine, phenylpropanolamine, pseudoephedrine, naphazoline hydrochloride, oxymetazoline hydrochloride, tetrahydrozoline hydrochloride, xylometazoline hydrochloride, and ethylnorepinephrine hydrochloride; (10) anticholinergic agents such as ipratropium bromide, tiotropium bromide, oxitropium bromide, aclindinium bromide, glycopyrrolate, (R,R)-glycopyrrolate, pirenzepine, and telenzepine; (11) β-adrenoceptor agonists such as metaproterenol, isoproterenol, isoprenaline, albuterol, formoterol (particularly the fumarate salt), salmeterol (particularly the xinafoate salt), terbutaline, orciprenaline, bitolterol mesylate, fenoterol, and pirbuterol, or methylxanthanines including theophylline and aminophylline, sodium cromoglycate; (12) insulin-like growth factor type I (IGF-1) mimetic; (13) glucocorticosteroids, especially inhaled glucocorticoid with reduced systemic side effects, such as prednisone, prednisolone, flunisolide, triamcinolone acetonide, beclomethasone dipropionate, budesonide, fluticasone propionate, ciclesonide and mometasone furoate; (14) kinase inhibitors such as inhibitors of the Janus Kinases (JAK 1 and/or JAK2 and/or JAK 3 and/or TYK2), p38 MAPK and IKK2; (15) B-cell targeting biologics such as rituximab (Rituxan®); (16) selective costimulation modulators such as abatacept (Orencia); (17) interleukin inhibitors, such as IL-1 inhibitor anakinra (Kineret) and IL-6 inhibitor tocilizumab (Actemra).

The present invention also provides for “triple combination” therapy, comprising a compound of Formula I or a pharmaceutically acceptable salt thereof together with beta₂-adrenoreceptor agonist and an anti-inflammatory corticosteroid. Preferably this combination is for treatment and/or prophylaxis of asthma, COPD or allergic rhinitis. The beta₂-adrenoreceptor agonist and/or the anti-inflammatory corticosteroid can be as described above and/or as described in WO 03/030939 A1. Representative examples of such a “triple” combination are a compound of Formula I or a pharmaceutically acceptable salt thereof in combination with the components of Advair® (salmeterol xinafoate and fluticasone propionate), Symbicort® (budesonide and formoterol fumarate), or Dulera® (mometasone furoate and formoterol).

For the treatment of cancer a compound of Formula I may be combined with one or more of an anticancer agents. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Such anti-cancer agents include, but are not limited to, the following: (1) estrogen receptor modulator such as diethylstibestral, tamoxifen, raloxifene, idoxifene, LY353381, LY117081, toremifene, fluoxymestero, and SH646; (2) other hormonal agents including aromatase inhibitors (e.g., aminoglutethimide, tetrazole anastrozole, letrozole and exemestane), luteinizing hormone release hormone (LHRH) analogues, ketoconazole, goserelin acetate, leuprolide, megestrol acetate and mifepristone; (3) androgen receptor modulator such as finasteride and other 5α-reductase inhibitors, nilutamide, flutamide, bicalutamide, liarozole, and abiraterone acetate; (4) retinoid receptor modulator such as bexarotene, tretinoin, 13-cis-retinoic acid, 9-cis-retinoic acid, α-difluoromethylomithine, ILX23-7553, trans-N-(4′-hydroxyphenyl) retinamide, and N-4-carboxyphenyl retinamide; (5) antiproliferative agent such asantisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, aminopterin, 5-flurouracil, floxuridine, methotrexate, leucovarin, hydroxyurea, thioguanine (6-TG), mercaptopurine (6-MP), cytarabine, pentostatin, fludarabine phosphate, cladribine (2-CDA), asparaginase, gemcitabine, alanosine, swainsonine, lometrexol, dexrazoxane, methioninase, and 3-aminopyridine-2-carboxaldehyde thiosemicarbazone; (6) prenyl-protein transferase inhibitor including farnesyl-protein transferase (FPTase), geranylgeranyl-protein transferase type I (GGPTase-I), and geranylgeranyl-protein transferase type-II (GGPTase-II, also called Rab GGPTase); (7) HMG-CoA reductase inhibitor such as lovastatin, simvastatin, pravastatin, atorvastatin, fluvastatin and rosuvastatin; (8) angiogenesis inhibitor such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α, interleukin-12, erythropoietin (epoietin-α), granulocyte-CSF (filgrastin), granulocyte, macrophage-CSF (sargramostim), pentosan polysulfate, cyclooxygenase inhibitors, steroidal anti-inflammatories, carboxyamidotriazole, combretastatin A-4, squalamine, 6-O-chloroacetyl-carbonyl)-fumagillol, thalidomide, angiostatin, troponin-1, angiotensin II antagonists, heparin, carboxypeptidase U inhibitors, and antibodies to VEGF, endostatin, ukrain, ranpimase, IM862, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RPI4610, NX31838, sulfated mannopentaose phosphate, and 3-[(2,4-dimethylpyrrol-5-yl)methylene]-2-indolinone (SU5416); (9) PPAR-γ agonists, PPAR-δ agonists, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, GI262570, PNU182716, DRF552926, 2-[(5,7-dipropyl-3-trifluoromethyl-1,2-benzisoxazol-6-yl)oxy]-2-methylpropionic acid (disclosed in U.S. Ser. No. 09/782,856), and (2R)-7-(3-(2-chloro-4-(4-fluorophenoxy)phenoxy)propoxy)-2-ethylchromane-2-carboxylic acid (disclosed in U.S. Ser. No. 60/235,708 and 60/244,697); (9) inhibitor of inherent multidrug resistance including inhibitors of p-glycoprotein (P-gp), such as LY335979, XR9576, OC144-093, R101922, VX853 and PSC833 (valspodar); (10) inhibitor of cell proliferation and survival signaling such as inhibitors of EGFR (for example gefitinib and erlotinib), inhibitors of ERB-2 (for example trastuzumab), inhibitors of IGF 1R such as MK-0646 (dalotuzumab), inhibitors of CD20 (rituximab), inhibitors of cytokine receptors, inhibitors of MET, inhibitors of PI3K family kinase (for example LY294002), serine/threonine kinases (including but not limited to inhibitors of Akt such as described in (WO 03/086404, WO 03/086403, WO 03/086394, WO 03/086279, WO 02/083675, WO 02/083139, WO 02/083140 and WO 02/083138), inhibitors of Raf kinase (for example BAY-43-9006), inhibitors of MEK (for example CI-1040 and PD-098059) and inhibitors of mTOR (for example Wyeth CCI-779 and Ariad AP23573); (11) a bisphosphonate such as etidronate, pamidronate, alendronate, risedronate, zoledronate, ibandronate, incadronate or cimadronate, clodronate, EB-1053, minodronate, neridronate, piridronate and tiludronate; (12) γ-secretase inhibitors, (13) agents that interfere with receptor tyrosine kinases (RTKs) including inhibitors of c-Kit, Eph, PDGF, Flt3 and c-Met; (14) agent that interferes with a cell cycle checkpoint including inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032; (15) BTK inhibitors such as PC132765, AVL-292 and AVL-101; (16) PARP inhibitors including iniparib, olaparib, AGO14699, ABT888 and MK4827; (16) ERK inhibitors; (17) mTOR inhibitors such as sirolimus, ridaforolimus, temsirolimus, everolimus; (18) cytotoxic/cytostatic agents.

“Cytotoxic/cytostatic agents” refer to compounds which cause cell death or inhibit cell proliferation primarily by interfering directly with the cell's functioning or inhibit or interfere with cell mytosis, including alkylating agents, tumor necrosis factors, intercalators, hypoxia activatable compounds, microtubule inhibitors/microtubule-stabilizing agents, inhibitors of mitotic kinesins, inhibitors of histone deacetylase, inhibitors of kinases involved in mitotic progression, antimetabolites; biological response modifiers; hormonal/anti-hormonal therapeutic agents, haematopoietic growth factors, monoclonal antibody targeted therapeutic agents, topoisomerase inhibitors, proteasome inhibitors and ubiquitin ligase inhibitors.

Examples of cytotoxic agents include, but are not limited to, sertenef, cachectin, chlorambucil, cyclophosphamide, ifosfamide, mechlorethamine, melphalan, uracil mustard, thiotepa, busulfan, carmustine, lomustine, streptozocin, tasonermin, lonidamine, carboplatin, altretamine, dacarbazine, procarbazine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum (II)]tetrachloride, diarizidinylspermine, arsenic trioxide, 1-(11-dodecylamino-10-hydroxyundecyl)-3,7-dimethylxanthine, zorubicin, doxorubicin, daunorubicin, idarubicin, anthracenedione, bleomycin, mitomycin C, dactinomycin, plicatomycin, bisantrene, mitoxantrone, pirarubicin, pinafide, valrubicin, amrubicin, antineoplaston, 3′-deamino-3′-morpholino-13-deoxo-10-hydroxycarminomycin, annamycin, galarubicin, elinafide, MEN10755, and 4-demethoxy-3-deamino-3-aziridinyl-4-methylsulphonyl-daunorubicin.

An example of a hypoxia activatable compound is tirapazamine.

Examples of proteasome inhibitors include but are not limited to lactacystin and bortezomib.

Examples of microtubule inhibitors/microtubule-stabilising agents include vincristine, vinblastine, vindesine, vinzolidine, vinorelbine, vindesine sulfate, 3′,4′-didehydro-4′-deoxy-8′-norvincaleukoblastine, podophyllotoxins (e.g., etoposide (VP-16) and teniposide (VM-26)), paclitaxel, docetaxol, rhizoxin, dolastatin, mivobulin isethionate, auristatin, cemadotin, RPR109881, BMS184476, vinflunine, cryptophycin, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797.

Some examples of topoisomerase inhibitors are topotecan, hycaptamine, irinotecan, rubitecan, 6-ethoxypropionyl-3′,4′-O-exo-benzylidene-chartreusin, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, 2,3-(methylenedioxy)-5-methyl-7-hydroxy-8-methoxybenzo[c]-phenanthridinium, 5-(3-aminopropylamino)-7,10-dihydroxy-2-(2-hydroxyethylaminomethyl)-6H-pyrazolo[4,5,1-de]acridin-6-one, N-[1-[2-(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, and dimesna.

Examples of inhibitors of mitotic kinesins include, but are not limited to inhibitors of KSP, inhibitors of MKLP1, inhibitors of CENP-E, inhibitors of MCAK, inhibitors of Kifl4, inhibitors of Mphosphl and inhibitors of Rab6-KIFL.

Examples of “histone deacetylase inhibitors” include, but are not limited to, vorinostat, trichostatin A, oxamflatin, PXD101, MG98, valproic acid and scriptaid.

“Inhibitors of kinases involved in mitotic progression” include, but are not limited to, inhibitors of aurora kinase, inhibitors of Polo-like kinases (PLK; in particular inhibitors of PLK-1), inhibitors of bub-1 and inhibitors of bub-R1. An example of an “aurora kinase inhibitor” is VX-680.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N6-[4-deoxy-4-[N2-[2,4-tetradecadienoyl]glycylamino]-L-glycero-B-L-manno-heptopyranosyl]adenine, aplidine, ecteinascidin, troxacitabine, aminopterin, 5-flurouracil, floxuridine, methotrexate, leucovarin, hydroxyurea, thioguanine (6-TG), mercaptopurine (6-MP), cytarabine, pentostatin, fludarabine phosphate, cladribine (2-CDA), asparaginase, gemcitabine, alanosine, swainsonine, lometrexol, dexrazoxane, methioninase, and 3-aminopyridine-2-carboxaldehyde thiosemicarbazone.

Non-limiting examples of suitable agents used in cancer therapy that may be combined with compounds of Formula I include, but are not limited to, abarelix; aldesleukin; alemtuzumab; alitretinoin; allopurinol; altretamine; amifostine; anastrozole; arsenic trioxide; asparaginase; azacitidine; bendamustine; bevacuzimab; bexarotene; bleomycin; bortezomib; busulfan; calusterone; capecitabine; carboplatin; carmustine; cetuximab; chlorambucil; cisplatin; cladribine; clofarabine; cyclophosphamide; cytarabine; dacarbazine; dactinomycin, actinomycin D; dalteparin; darbepoetin alfa; dasatinib; daunorubicin; degarelix; denileukin diftitox; dexrazoxane; docetaxel; doxorubicin; dromostanolone propionate; eculizumab; Elliott's B Solution; eltrombopag; epirubicin; epoetin alfa; erlotinib; estramustine; etoposide phosphate; etoposide; everolimus; exemestane; filgrastim; floxuridine; fludarabine; fluorouracil; fulvestrant; gefitinib; gemcitabine; gemtuzumab ozogamicin; goserelin acetate; histrelin acetate; hydroxyurea; ibritumomab tiuxetan; idarubicin; ifosfamide; imatinib mesylate; interferon alfa 2a; interferon alfa-2b; irinotecan; ixabepilone; lapatinib; lenalidomide; letrozole; leucovorin; leuprolide acetate; levamisole; lomustine; meclorethamine, nitrogen mustard; megestrol acetate; melphalan, L-PAM; mercaptopurine; mesna; methotrexate; methoxsalen; mitomycin C; mitotane; mitoxantrone; nandrolone phenpropionate; nelarabine; nilotinib; Nofetumomab; ofatumumab; oprelvekin; oxaliplatin; paclitaxel; palifermin; pamidronat; panitumumab; pazopanib; pegademase; pegaspargase; Pegfilgrastim; pemetrexed disodium; pentostatin; pipobroman; plerixafor; plicamycin, mithramycin); porfimer sodium; pralatrexate; procarbazine; quinacrine; Rasburicase; raloxifene hydrochloride; Rituximab; romidepsin; romiplostim; sargramostim; sargramostim; satraplatin; sorafenib; streptozocin; sunitinib maleate; tamoxifen; temozolomide; temsirolimus; teniposide; testolactone; thioguanine; thiotepa; topotecan; toremifene; tositumomab; trastuzumab; tretinoin; uracil mustard; valrubicin; vinblastine; vincristine; vinorelbine; vorinostat; and zoledronate.

It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates, to optimise the activity and/or stability and/or physical characteristics, such as solubility, of the therapeutic ingredient. It will be clear also that, where appropriate, the therapeutic ingredients may be used in optically pure form.

The combinations referred to above may conveniently be presented for use in the form of a pharmaceutical composition and thus pharmaceutical compositions comprising a combination as defined above together with a pharmaceutically acceptable diluent, carrier or excipient represent a further aspect of the invention. These combinations are of particular interest in respiratory diseases and are conveniently adapted for inhaled or intranasal delivery.

The individual compounds of such combinations may be administered either sequentially or simultaneously in separate or combined pharmaceutical compositions. Preferably, the individual compounds will be administered simultaneously in a combined pharmaceutical composition. Appropriate doses of known therapeutic agents will be readily appreciated by those skilled in the art.

### Pharmaceutical Compositions

While it is possible that, for use in therapy, a compound of Formula I, as well as salts, solvates and physiological functional derivatives thereof, may be administered as the raw chemical, it is possible to present the active ingredient as a pharmaceutical composition. Accordingly, the invention further provides a pharmaceutical composition which comprises a compound of Formula I and salts, solvates and physiological functional derivatives thereof, and one or more pharmaceutically acceptable carriers, diluents, or excipients. The compounds of the Formula I and salts, solvates and physiological functional derivatives thereof, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical composition including admixing a compound of the Formula I, or salts, solvates and physiological functional derivatives thereof, with one or more pharmaceutically acceptable carriers, diluents or excipients.

### Routes of Administration

Pharmaceutical compositions of the present invention may be presented in unit dose forms containing a predetermined amount of active ingredient per unit dose. Such a unit may contain, for example, 5 μg to 1 g, preferably 1 mg to 700 mg, more preferably 5 mg to 100 mg of a compound of the Formula I, depending on the condition being treated, the route of administration and the age, weight and condition of the patient. Such unit doses may therefore be administered more than once a day. Preferred unit dosage compositions are those containing a daily dose or sub-dose (for administration more than once a day), as herein above recited, or an appropriate fraction thereof, of an active ingredient. Furthermore, such pharmaceutical compositions may be prepared by any of the methods well known in the pharmacy art.

Pharmaceutical compositions of the present invention may be adapted for administration by any appropriate route, for example by the oral (including buccal or sublingual), rectal, topical, inhaled, nasal, ocular, sublingual, subcutaneous, local or parenteral (including intravenous and intramuscular) route, and the like, all in unit dosage forms for administration. Such compositions may be prepared by any method known in the art of pharmacy, for example by bringing into association the active ingredient with the carrier(s) or excipient(s). Dosage forms include tablets, troches, dispersions, suspensions, solutions, capsules, creams, ointments, aerosols, and the like.

In a further embodiment, the present invention provides a pharmaceutical composition adapted for administration by the oral route, for treating, for example, rheumatoid arthritis.

In a further embodiment, the present invention provides a pharmaceutical composition adapted for administration by the nasal route, for treating, for example, allergic rhinitis.

In a further embodiment, the present invention provides a pharmaceutical composition adapted for administration by the inhaled route, for treating, for example, asthma, Chronic Obstructive Pulmonary disease (COPD) or Acute Respiratory Distress Syndrome (ARDS).

In a further embodiment, the present invention provides a pharmaceutical composition adapted for administration by the ocular route, for treating, diseases of the eye, for example, conjunctivitis.

In a further embodiment, the present invention provides a pharmaceutical composition adapted for administration by the parenteral (including intravenous) route, for treating, for example, cancer.

For parenteral administration, the pharmaceutical composition of the invention may be presented in unit-dose or multi-dose containers, e.g. injection liquids in predetermined amounts, for example in sealed vials and ampoules, and may also be stored in a freeze dried (lyophilized) condition requiring only the addition of sterile liquid carrier, e.g. water, prior to use.

Mixed with such pharmaceutically acceptable auxiliaries, e.g. as described in the standard reference, Gennaro, A. R. et al., Remington: The Science and Practice of Pharmacy (20th Edition., Lippincott Williams & Wilkins, 2000, see especially Part 5: Pharmaceutical Manufacturing), the active agent may be compressed into solid dosage units, such as pills, tablets, or be processed into capsules or suppositories. By means of pharmaceutically acceptable liquids the active agent can be applied as a fluid composition, e.g. as an injection preparation, in the form of a solution, suspension, emulsion, or as a spray, e.g. a nasal spray.

For making solid dosage units, the use of conventional additives such as fillers, colorants, polymeric binders and the like is contemplated. In general any pharmaceutically acceptable additive which does not interfere with the function of the active compounds can be used. Suitable carriers with which the active agent of the invention can be administered as solid compositions include lactose, starch, cellulose derivatives and the like, or mixtures thereof, used in suitable amounts. For parenteral administration, aqueous suspensions, isotonic saline solutions and sterile injectable solutions may be used, containing pharmaceutically acceptable dispersing agents and/or wetting agents, such as propylene glycol or butylene glycol.

Pharmaceutical compositions of the present invention which are adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit compositions for oral administration can be microencapsulated. The formulation can also be prepared to prolong or sustain the release, for example, by coating or embedding particulate material in polymers, wax or the like.

The compounds of Formula I, and salts, solvates and physiological functional derivatives thereof, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of Formula I and salts, solvates and physiological functional derivatives thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Dosage forms for inhaled administration may conveniently be formulated as aerosols or dry powders.

For compositions suitable and/or adapted for inhaled administration, it is preferred that the compound or salt of Formula I is in a particle-size-reduced form, and more preferably the size-reduced form is obtained or obtainable by micronisation. The preferable particle size of the size-reduced (e.g. micronised) compound or salt or solvate is defined by a D50 value of about 0.5 to about 10 microns (for example as measured using laser diffraction).

Aerosol formulations, e.g. for inhaled administration, can comprise a solution or fine suspension of the active substance in a pharmaceutically acceptable aqueous or non-aqueous solvent. Aerosol formulations can be presented in single or multidose quantities in sterile form in a sealed container, which can take the form of a cartridge or refill for use with an atomising device or inhaler. Alternatively the sealed container may be a unitary dispensing device such as a single dose nasal inhaler or an aerosol dispenser fitted with a metering valve (metered dose inhaler) which is intended for disposal once the contents of the container have been exhausted.

Where the dosage form comprises an aerosol dispenser, it preferably contains a suitable propellant under pressure such as compressed air, carbon dioxide or an organic propellant such as a hydrofluorocarbon (HFC). Suitable HFC propellants include 1,1,1,2,3,3,3-heptafluoropropane and 1,1,1,2-tetrafluoroethane. The aerosol dosage forms can also take the form of a pump-atomiser. The pressurised aerosol may contain a solution or a suspension of the active compound. This may require the incorporation of additional excipients e.g. co-solvents and/or surfactants to improve the dispersion characteristics and homogeneity of suspension formulations. Solution formulations may also require the addition of co-solvents such as ethanol. Other excipient modifiers may also be incorporated to improve, for example, the stability and/or taste and/or fine particle mass characteristics (amount and/or profile) of the formulation.

For pharmaceutical compositions suitable and/or adapted for inhaled administration, it is preferred that the pharmaceutical composition is a dry powder inhalable composition. Such a composition can comprise a powder base such as lactose, glucose, trehalose, mannitol or starch, the compound of Formula I or salt or solvate thereof (preferably in particle-size-reduced form, e.g. in micronised form), and optionally a performance modifier such as L-leucine or another amino acid, and/or metals salts of stearic acid such as magnesium or calcium stearate. Preferably, the dry powder inhalable composition comprises a dry powder blend of lactose and the compound of Formula I or salt thereof. The lactose is preferably lactose hydrate e.g. lactose monohydrate and/or is preferably inhalation-grade and/or fine-grade lactose. Preferably, the particle size of the lactose is defined by 90% or more (by weight or by volume) of the lactose particles being less than 1000 microns (micrometres) (e.g. 10-1000 microns e.g. 30-1000 microns) in diameter, and/or 50% or more of the lactose particles being less than 500 microns (e.g. 10-500 microns) in diameter. More preferably, the particle size of the lactose is defined by 90% or more of the lactose particles being less than 300 microns (e.g. 10-300 microns e.g. 50-300 microns) in diameter, and/or 50% or more of the lactose particles being less than 100 microns in diameter. Optionally, the particle size of the lactose is defined by 90% or more of the lactose particles being less than 100-200 microns in diameter, and/or 50% or more of the lactose particles being less than 40-70 microns in diameter. It is preferable that about 3 to about 30% (e.g. about 10%) (by weight or by volume) of the particles are less than 50 microns or less than 20 microns in diameter. For example, without limitation, a suitable inhalation-grade lactose is E9334 lactose (10% fines) (Borculo Domo Ingredients, Hanzeplein 25, 8017 J D Zwolle, Netherlands).

Optionally, in particular for dry powder inhalable compositions, a pharmaceutical composition for inhaled administration can be incorporated into a plurality of sealed dose containers (e.g. containing the dry powder composition) mounted longitudinally in a strip or ribbon inside a suitable inhalation device. The container is rupturable or peel-openable on demand and the dose of e.g. the dry powder composition can be administered by inhalation via the device such as the DISKUS® device (GlaxoSmithKline). Other dry powder inhalers are well known to those of ordinary skill in the art, and many such devices are commercially available, with representative devices including Aerolizer® (Novartis), Airmax™ (IVAX), ClickHaler® (Innovata Biomed), Diskhaler® (GlaxoSmithKline), Accuhaler (GlaxoSmithKline), Easyhaler® (Orion Pharma), Eclipse™ (Aventis), FlowCaps® (Hovione), Handihaler® (Boehringer Ingelheim), Pulvinal® (Chiesi), Rotahaler® (GlaxoSmithKline), SkyeHaler™ or Certihaler™ (SkyePharma), Twisthaler (Schering-Plough), Turbuhaler® (AstraZeneca), Ultrahaler® (Aventis), and the like.

Dosage forms for ocular administration may be formulated as solutions or suspensions with excipients suitable for ophthalmic use.

Dosage forms for nasal administration may conveniently be formulated as aerosols, solutions, drops, gels or dry powders.

Pharmaceutical compositions adapted for administration by inhalation include fine particle dusts or mists, which may be generated by means of various types of metered, dose pressurized aerosols, nebulizers or insufflators.

For pharmaceutical compositions suitable and/or adapted for intranasal administration, the compound of Formula I or a pharmaceutically acceptable salt or solvate thereof may be formulated as a fluid formulation for delivery from a fluid dispenser. Such fluid dispensers may have, for example, a dispensing nozzle or dispensing orifice through which a metered dose of the fluid formulation is dispensed upon the application of a user-applied force to a pump mechanism of the fluid dispenser. Such fluid dispensers are generally provided with a reservoir of multiple metered doses of the fluid formulation, the doses being dispensable upon sequential pump actuations. The dispensing nozzle or orifice may be configured for insertion into the nostrils of the user for spray dispensing of the fluid formulation into the nasal cavity. A fluid dispenser of the aforementioned type is described and illustrated in WO-A-2005/044354, the entire content of which is hereby incorporated herein by reference. The dispenser has a housing which houses a fluid discharge device having a compression pump mounted on a container for containing a fluid formulation. The housing has at least one finger-operable side lever which is movable inwardly with respect to the housing to cam the container upwardly in the housing to cause the pump to compress and pump a metered dose of the formulation out of a pump stem through a nasal nozzle of the housing. A particularly preferred fluid dispenser is of the general type illustrated in FIGS. 30-40 of WO-A-2005/044354.

The invention further includes a pharmaceutical composition of a compound of Formula I or pharmaceutically acceptable salts thereof, as hereinbefore described, in combination with packaging material suitable for said composition, said packaging material including instructions for the use of the composition for the use as hereinbefore described.

The following are examples of representative pharmaceutical dosage forms for the compounds of this invention:

| Injectable Suspension (I.M.) | mg/ml |
|---|---|
| Compound of Formula I | 10 |
| Methylcellulose | 5.0 |
| Tween 80 | 0.5 |
| Benzyl alcohol | 9.0 |
| Benzalkonium chloride | 1.0 |

Water for injection to a total volume of 1 ml

| Tablet | mg/tablet |
|---|---|
| Compound of Formula I | 25 |
| Microcrystalline Cellulose | 415 |
| Providone | 14.0 |
| Pregelatinized Starch | 43.5 |
| Magnesium Stearate | 2.5 |
| | 500 |

| Capsule | mg/capsule |
|---|---|
| Compound of Formula I | 25 |
| Lactose Powder | 573.5 |
| Magnesium Stearate | 1.5 |
| | 600 |

| Aerosol | Per canister |
|---|---|
| Compound of Formula I | 24 mg |
| Lecithin, NF Liquid Concentrate | 1.2 mg |
| Trichlorofluoromethane, NF | 4.025 gm |
| Dichlorodifluoromethane, NF | 12.15 gm |

It will be appreciated that when the compound of the present invention is administered in combination with other therapeutic agents normally administered by the inhaled, intravenous, oral or intranasal route, that the resultant pharmaceutical composition may be administered by the same routes.

It should be understood that in addition to the ingredients particularly mentioned above, the compositions may include other agents conventional in the art having regard to the type of formulation in question, for example those suitable for oral administration may include flavoring agents.

A therapeutically effective amount of a compound of the present invention will depend upon a number of factors including, for example, the age and weight of the animal, the precise condition requiring treatment and its severity, the particular compound having Formula I, the nature of the formulation, and the route of administration, and will ultimately be at the discretion of the attendant physician or veterinarian. However, an effective amount of a compound of Formula I for the treatment of diseases or conditions associated with inappropriate Btk activity, will generally be in the range of 5 μg to 100 mg/kg body weight of recipient (mammal) per day and more usually in the range of 5 μg to 10 mg/kg body weight per day. This amount may be given in a single dose per day or more usually in a number (such as two, three, four, five or six) of sub-doses per day such that the total daily dose is the same. An effective amount of a salt or solvate, thereof, may be determined as a proportion of the effective amount of the compound of Formula I per se.

In general parenteral administration requires lower dosages than other methods of administration which are more dependent upon absorption. However, a dosage for humans preferably contains 0.0001-25 mg of a compound of Formula I or pharmaceutically acceptable salts thereof per kg body weight. The desired dose may be presented as one dose or as multiple subdoses administered at appropriate intervals throughout the day, or, in case of female recipients, as doses to be administered at appropriate daily intervals throughout the menstrual cycle. The dosage as well as the regimen of administration may differ between a female and a male recipient.

### General Synthesis

The compounds of the present invention can be prepared by methods well known in the art of organic chemistry. See, for example, J. March, ‘Advanced Organic Chemistry’ 4^{th} Edition, John Wiley and Sons. During synthetic sequences it may be necessary and/or desirable to protect sensitive or reactive groups on any of the molecules concerned. This is achieved by means of conventional protecting groups, such as those described in T. W. Greene and P. G. M. Wutts ‘Protective Groups in Organic Synthesis’ 3^{rd} Edition, John Wiley and Sons, 1999. The protective groups are optionally removed at a convenient subsequent stage using methods well known in the art.

The products of the reactions are optionally isolated and purified, if desired, using conventional techniques, but not limited to, filtration, distillation, crystallization, chromatography and the like. Such materials are optionally characterized using conventional means, including physical constants and spectral data.

The compounds of Formula I can be prepared by the general synthetic routes shown in the schemes below.

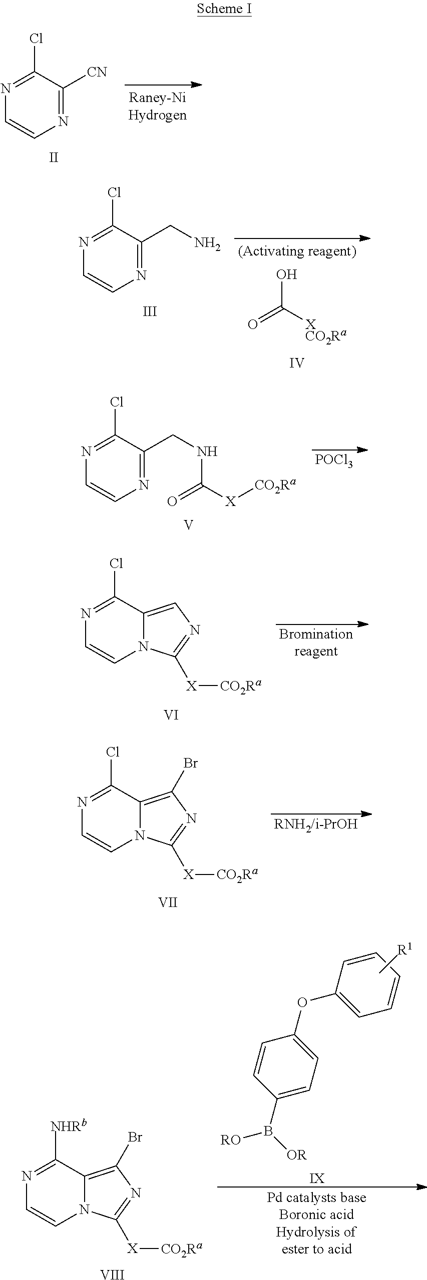

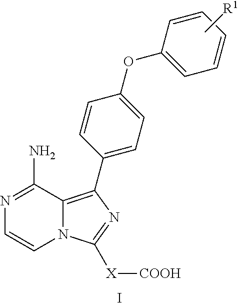

Reduction of 3-chloropyrazine-2-carbonitrile (II) can be accomplished by hydrogenation in the presence of a suitable catalyst system and solvent, for example Raney-Nickel ethanol to provide (3-chloropyrazin-2-yl)methanamine (III). This amine can then be reacted with the diacid monoester (IV). The reaction of IV can be carried out in a solvent such as DMF, THF or DCM in the presence of a base such as DIPEA, N-methylmorpholine, 4-DMAP or triethylamine and in the presence of a coupling reagent such as PyBOP, TBTU, EDCI or HATU to form N-((3-chloropyrazin-2-yl)methyl)amide (V). Cyclization chloropyrazine (V) can be performed using condensation reagents like phosphorousoxychloride under heating conditions to provide the 8-chloroimidazo[1,5-a]pyrazine derivatives VI. Subsequent bromination can be accomplished using bromine or N-bromosuccinimide in a suitable solvent like DCM or DMF at appropriate temperature to obtain compounds of formula VII. 8-Aminoimidazo[1,5-a]pyrazine derivatives (VIII) can be prepared from compounds VII using ammonia (gas) in isopropanol at elevated temperature in a pressure vessel (>4 atm) or with primary amine (such as dimethoxybenzylamine) under heating. Compounds of formula I can be prepared from compounds of formula VIII using an appropriate boronic acid or pinacol ester (IX), in the presence of a suitable palladium catalyst system, for example bis(diphenylphosphino)ferrocene palladium(II)chloride complex or tetrakis(triphenylphosphine)palladium(0) in the presence of an inorganic base like potassium carbonate, cesium carbonate or potassium phosphate in a suitable solvent system like combinations of dioxane and water. Palladium catalysts and conditions to form either the pinacol esters or to couple the boronic acids or pinacol esters with the 1-bromoimidazo[1,5-a]pyrazin-8-amine are well known to the skilled organic chemist—see, for example, Ei-ichi Negishi (Editor), Armin de Meijere (Associate Editor), Handbook of Organopalladium Chemistry for Organic Synthesis, John Wiley and Sons, 2002. The ester usually is hydrolyzed during the Suzuki coupling with water as co-solvent, otherwise one additional step for the hydrolysis of ester under conventional basic or acid conditions. The diacid mono esters IV are commercially available or can be readily prepared using methods well known to the skilled organic chemist.

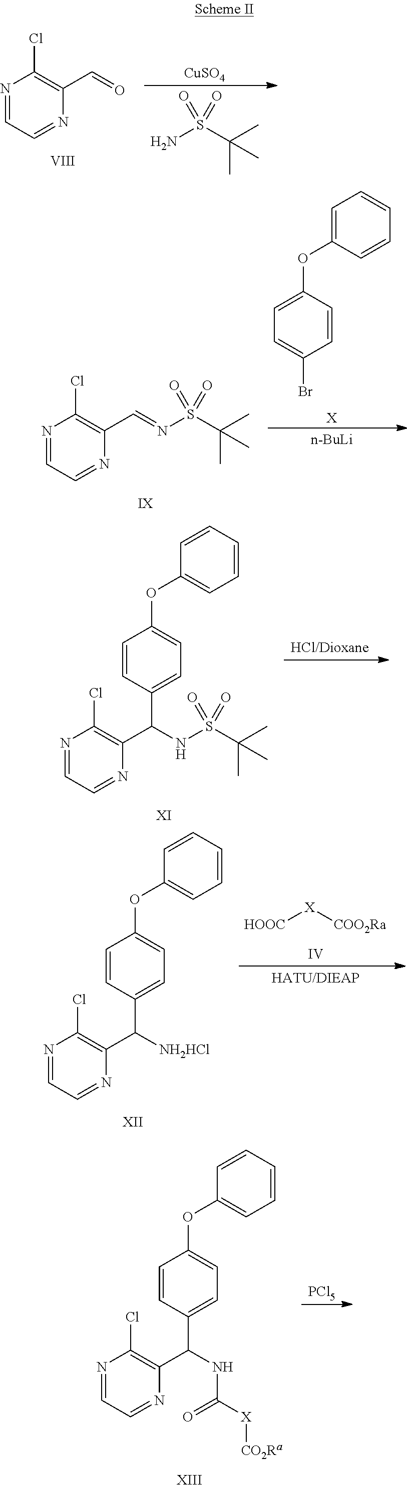

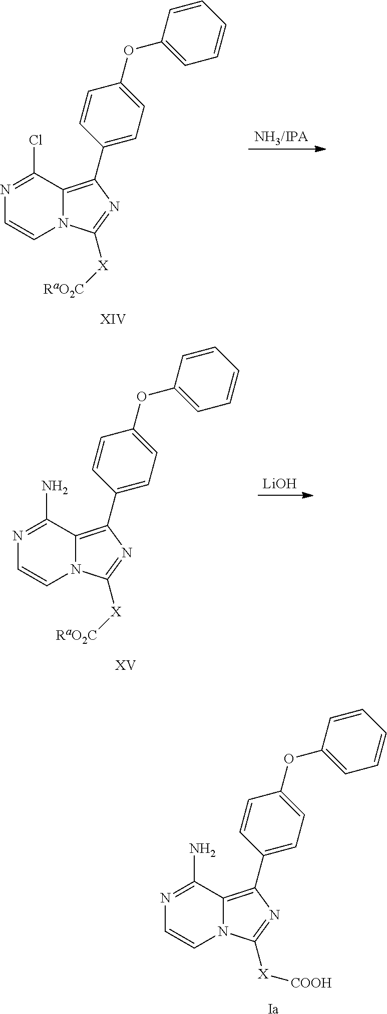

An alternative route is shown in Scheme II for the preparation of compound with formula Ia. The aldehyde VIII was converted to sulfonylimine IX by reacting with tertbutylsulfonamide with cuperic sulfate. The addition of the phenoxyphenyl lithium freshly prepared by reacting butyl lithium with phenoxyphenyl bromide generates sulfonylamide XI. The sulfonylamide is then hydrolyzed to amine hydrochloride salt XII after treatment with HCl in dioxane. The coupling of diacid mono ester IV with XII provide the coupled product XIII, which was then treated with phosphorus pentachloride to cyclized to imidazopyrazine compound XIV. The 8-chloro was converted to amino by treating XIV with ammonia in isopropanol at high temperature to provide compound XV. Hydrolysis of the ester in XV generates the final product Ia.

The following abbreviations are used throughout the application with respect to chemical terminology:
HATU O-(7-Azabenzotriazol-1-yl)-1,1,3,3-tetramethyluroniumhexafluoro phosphate
Cbz Benzyloxycarbonyl
DMF N,N-Dimethylformamide
DCM Dichloromethane
EtOAc Ethyl acetate
DIPEA N,N-Diisopropylethylamine
THF Tetrahydrofuran
EtOH Ethanol
EDCI.HCl 1-(3-Dimethylaminopropyl)-3-ethylcarbodiimide hydrochloride
4-DMAP 4-Dimethylaminopyridine
PyBOP O-Benzotriazole-1-yl-oxy-trispyrrolidinophosphonium hexafluorophosphate
TBTU O-Benzotriazol-1-yl-N,N,N′,N′-tetramethyluronium tetrafluoroborate
HBr Hydrogen bromide
HCl Hydrogen chloride
HOAc Acetic acid
POCl₃ Phosphorous oxychloride
HPLC High Pressure Liquid Chromatography
UPLC Ultra Performance Liquid Chromatography
LiHMDS Lithium hexamethyldisilazide
MeOH Methanol
DCM Dichloromethane
n-BuLi n-Butyllithium
CO₂ Carbondioxide
NaHCO₃ Sodium bicarbonate
K₃PO₄ Potassium phosphate
P(Cy)₃ Tricyclohexylphosphine
Pd(OAc)₂ Palladium(II) acetate
Na₂SO₄ Sodium sulfate
Na₂CO₃ Sodium carbonate
DAST Diethylaminosulfur trifluoride
Cs₂CO₃ Cesium carbonate
Et₂O Diethylether
Na₂S₂O₃ Sodium thiosulfate
Na₂S₂O₄ Sodium hydrosulfite
NaCNBH₃ Sodium cyanoborohydride
NH₄Cl Ammonium chloride
MgSO₄ Magnesium sulfate
LiOH Lithium hydroxide
IPA Isopropylamine
TFA Trifluoroacetic acid
Cbz-Cl Benzylchloroformate
PE Petroleum ether
EA Ethyl acetate
NaHMDS Sodium hexamethyldisilazide
10% Pd/C 10% Palladium on carbon
TEA Triethylamine
CDI 1,1′-Carbonyl diimidazole
DMI 1,3-Dimethyl-2-imidazolidinone
NBS N-Bromosuccinimide
i-PrOH 2-Propanol
K₂CO₃ Potassium carbonate
Pd(dppf)Cl₂ 1,1′-Bis(diphenylphosphino)ferrocene palladium (II) chloride, complex with dichloromethane
Et₃N Triethylamine
2-BuOH 2-Butanol
LCMS Liquid Chromatography/Mass Spectrometry
MeCN Acetonitrile
NH₃ Ammonia
CD₃I Trideuteromethyl iodide
CD₃OD Tetradeuteromethanol
CH₃I Iodomethane
CBr₄ Carbon tetrabromide
Tris-HCl Tris(hydroxymethyl)aminomethane hydrochloride
MgCl₂ Magnesium chloride
NaN₃ Sodium azide
DTT Dithiothreitol
DMSO Dimethyl sulfoxide
IMAP Immobilized Metal Ion Affinity-Based Fluorescence Polarization
ATP Adenosine triphosphate
MnCl₂ Manganese(II) chloride
DMA Dimethylacetamide
IPA Isopropyl alcohol
TPP triphenylphosphine
DIAD Diisopropyl azodicarboxylate
DMB 2,4-dimethoxybenzyl
DCE Dichloroethane
DEAD Diethyl azodicarboxylate
ACN Acetonitrile
RT (rt) Room Temperature
Aq Aqueous
EtOH Ethanol
MPLC Medium Pressure Liquid Chromatography
Xantphos 4,5-Bis(diphenylphosphino)-9,9-dimethylxanthene
X-phos 2-Dicyclohexylphosphino-2′,4′,6′-triisopropylbiphenyl
EA Ethyl Acetate
NBS N-bromosuccinimide

The invention is illustrated by the following examples.

### EXAMPLES

The following examples are illustrative embodiments of the invention, not limiting the scope of the invention in any way. Reagents are commercially available or are prepared according to procedures in the literature.

Mass Spectrometry: Electron Spray spectra were recorded on the Applied Biosystems API-165 single quad mass spectrometer in alternating positive and negative ion mode using Flow Injection. The mass range was 120-2000 Da. and scanned with a step rate of 0.2 Da. and the capillary voltage was set to 5000 V. N₂ gas was used for nebulisation.

LC-MS spectrometer (Waters) Detector: PDA (200-320 nm), Mass detector: ZQ and Eluent: A: acetonitrile with 0.05% trifluoroacetic acid, B: acetronitrile/water=1/9 (v/v) with 0.05% trifluoroacetic acid.

### Method A: LC-MS

| Column | Ascentis Express C18, 100 × 3.0 mm, 2.7 μm | |
|---|---|---|
| | A: H₂O (0.1% TFA) | |
| Mobile Phase | B: MeCN (0.05% TFA) | |
| | Stop Time: 5.0 min | |
| | Time (min) | B % |
| Gradient | 0.00 | 10 |
| | 3.50 | 99 |
| | 4.99 | 99 |
| | 5.00 | 10 |

| | |
|---|---|
| Sample injection | 2 μl |
| volume | |
| Flow Rate | 1.00 ml/min |
| Wavelength | 220 nm |
| Oven Tem. | 50° C. |
| MS polarity | ESI POS |

### Method B: LC-MS

| Column | Ascentis Express C18, 50 × 2.1 mm, 5 μm | |
|---|---|---|
| | A: H₂O (0.1% TFA) | |
| Mobile Phase | B: MeCN (0.05% TFA) | |
| | Stop Time: 2.0 min | |
| | Time (min) | B % |
| Gradient | 0 | 10 |
| | 0.8 | 99 |
| | 1.99 | 99 |
| | 2.00 | 10 |

| | |
|---|---|
| Sample injection | 2 μl |
| volume | |
| Flow Rate | 1.25 ml/min |
| Wavelength | 220 nm |
| Oven Temp. | 50° C. |
| MS polarity | ESI POS |

### Method C:

Sample Info: Easy-Access Method: ‘1-Short_TFA_Pos’
Method Info: B222 Column Agilent SBC (3.0×50 mm, 1.8 m); Flow 1.0 mL/min; solvent A: H₂O-0.1% TFA;
solvent B: MeCN-0.1% TFA;

### GRADIENT TABLE: 0 min:10% B, 0.3 min:10% B, 1.5 min: 95% B, 2.70 min: 95% B, 2.76 min: 10% B

stop time 3.60 min, PostTime 0.70 min.

### Method D:

### Sample Info: Easy-Access Method: ‘1_Fast’

Method Info: A330 Column Agilent Zorbax SB-C18 (2.1×30 mm, 3.5 m); Flow 2.0 mL/min; solvent A: H₂O-0.1% TFA;
solvent B: MeCN-0.1% TFA;
GRADIENT TABLE: 0.01 min:10% B, 1.01 min:95% B, 1.37 min:95% B, 1.38 min:10% B, stop time 1.7 min, PostTime=OFF

### INTERMEDIATES

### Intermediate 1 and 2

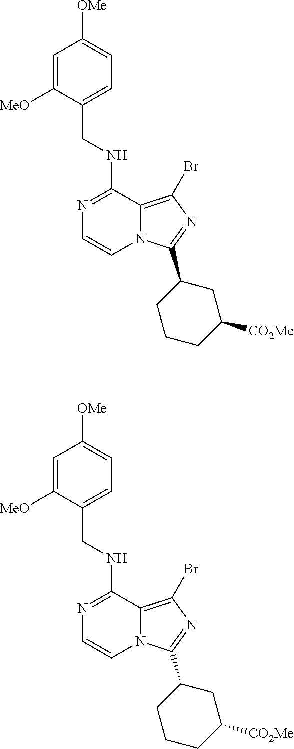 [SMILES: COC(=O)[C@H]1CCC[C@@H](c2nc(Br)c3c(NCc4ccc(OC)cc4OC)nccn23)C1 ; COC(=O)[C@@H]1CCC[C@H](c2nc(Br)c3c(NCc4ccc(OC)cc4OC)nccn23)C1]

### (1S,3R)-methyl 3-(1-bromo-8-((2,4-dimethoxybenzyl)amino)imidazo[1,5-a]pyrazin-3-yl)cyclohexanecarboxylate

### (1R,3S)-methyl 3-(1-bromo-8-((2,4-dimethoxybenzyl)amino)imidazo[1,5-a]pyrazin-3-yl)cyclohexanecarboxylate

### Step 1: cis-methyl 3-(((3-chloropyrazin-2-yl)methyl)carbamoyl)cyclohexanecarboxylate

HATU (12.18 g, 32.0 mmol) was added to a stirred mixture of (3-chloropyrazin-2-yl)methanamine (4.22 g, 29.4 mmol), (1R,3S)-3-(methoxycarbonyl)cyclohexanecarboxylic acid (4.97 g, 26.7 mmol) and DIPEA (13.98 ml, 80 mmol) in DMF (100 ml) and the mixture was stirred at room temperature for 3 h. The reaction mixture was then diluted with water, extracted with EA (3×), washed with water, brine and concentrated. The residue was purified by column chromatography on silica gel (120 g), eluting with EtOAc/isohexane (1/1) to cis-methyl 3-(((3-chloropyrazin-2-yl)methyl)carbamoyl)cyclohexanecarboxylate. LC-MS: C₁₄H₁₈ClN₃O₃, found [M+1]⁺: 312.1.

### Step 2: cis-methyl 3-(8-chloroimidazo[1,5-a]pyrazin-3-yl)cyclohexanecarboxylate

POCl₃ (0.963 ml, 10.33 mmol) was added to a stirred mixture of 3-(((3-chloropyrazin-2-yl)methyl)carbamoyl)cyclohexanecarboxylate (920 mg, 2.95 mmol) in acetonitrile (50 m) and the mixture was stirred at 70° C. for 45 min. The reaction mixture was then concentrated and the residue cis-methyl 3-(8-chloroimidazo[1,5-a]pyrazin-3-yl)cyclohexanecarboxylate which was used direct to next step without further purification. LC-MS: C₁₄H₁₆ClN₃O₂, found [M+1]⁺: 294.1.

### Step 3: cis-methyl 3-(1-bromo-8-chloroimidazo[1,5-a]pyrazin-3-yl)cyclohexanecarboxylate

NBS (578 mg, 3.25 mmol) was added to a stirred mixture of cis-methyl 3-(8-chloroimidazo[1,5-a]pyrazin-3-yl)cyclohexanecarboxylate (867 mg, 2.95 mmol) in DMF (20 ml) and the mixture was stirred at room temperature for 1 h. The reaction mixture was the diluted with EtOAc, washed with sat. NaHCO₃, water, dried over MgSO₄, filtered and concentrated. The residue was purified by column chromatography on silica gel (ISco, 80 g), eluting with EtOAc/isohexane (3/2) to give cis-methyl 3-(1-bromo-8-chloroimidazo[1,5-a]pyrazin-3-yl)cyclohexanecarboxylate. LC-MS: C₁₄H₁₅BrClN₃O₂, found [M+1]⁺, [M+2]⁺: 372.0, 374.0.

### Step 4: (1S,3R)-methyl 3-(1-bromo-8-((2,4-dimethoxybenzyl)amino)imidazo[1,5-a]pyrazin-3-yl)cyclohexanecarboxylate

(2,4-dimethoxyphenyl)methanamine (1.356 ml, 8.92 mmol) was added to a stirred mixture of cis-methyl 3-(1-bromo-8-chloroimidazo[1,5-a]pyrazin-3-yl)cyclohexanecarboxylate (950 mg, 2.55 mmol), and N-ethyl-N-isopropylpropan-2-amine (1.558 ml, 8.92 mmol). The mixture was stirred at 0° C. to room temperature overnight. The reaction mixture was concentrated. The residue was purified by column chromatography on silica gel, eluting with EtOAc/isohexane (10/1 to 5/1) to give methyl 3-(1-bromo-8-((2,4-dimethoxybenzyl)amino)imidazo[1,5-a]pyrazin-3-yl)cyclohexanecarboxylate. LC-MS: C23H27BrN4O4, found [M+1]⁺, [M+2]⁺: 503.1, 505.1. The cis enantiomers were separated by SFC (OJ-H column, 40% EtOH/CO₂) to afford two enantiomer: (1S,3R)-methyl 3-(1-bromo-8-((2,4-dimethoxybenzyl)amino)imidazo[1,5-a]pyrazin-3-yl)cyclohexanecarboxylate (El, retention time: 6.01 min) and (1R,3S)-methyl 3-(1-bromo-8-((2,4-dimethoxybenzyl)amino)imidazo[1,5-a]pyrazin-3-yl)cyclohexanecarboxylate (E2, retention time: 9.56 min).

### Intermediate 2

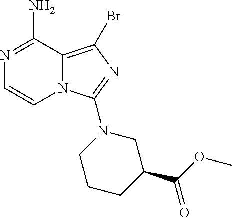 [SMILES: COC(=O)[C@H]1CCCN(c2nc(Br)c3c(N)nccn23)C1]

### (S)-methyl 1-(8-amino-1-bromoimidazo[1,5-a]pyrazin-3-yl)piperidine-3-carboxylate

To a solution of (R)-methyl piperidine-3-carboxylate (613 mg, 2.1 mmol), and 1,3-dibromoimidazo[1,5-a]pyrazin-8-amine (580 mg, 2 mmol) in NMP (2.5 mL) was added DIEA (2.25 g, 17.5 mmol). The mixture was stirred at 150° C. under microwave for 0.5 hour. After cooling to room temperature, the mixture was added H₂O (10 mL), extracted by EA (10 mL). The organic layer was dried over anhydrous Na₂SO₄ and concentrated in vacuo. And then the residue was purified by pre-HPLC to give (S)-methyl1-(8-amino-1-bromoimidazo[1,5-a]pyrazin-3-yl)piperidine-3-carboxylate. 1H NMR (400 MHz, DMSO-d6) δ=7.66 (d, J=5.5 Hz, 1H), 6.94 (d, J=5.5 Hz, 1H), 3.65-3.59 (m, 1H), 3.30 (d, J=12.0 Hz, 2H), 3.00-2.91 (m, 1H), 2.14 (br. s., 1H), 1.71 (d, J=4.0 Hz, 2H), 1.40-1.29 (m, 1H), 1.14 (s, 3H)

### Intermediate 4, 5, 6, 7

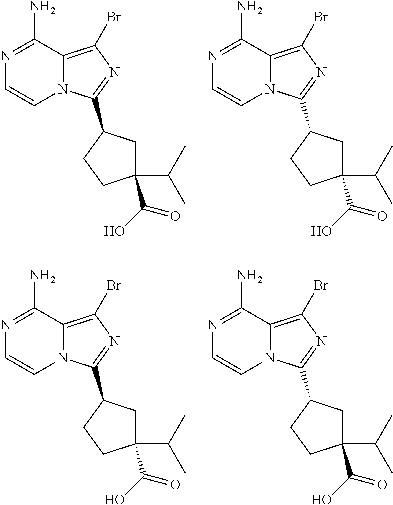 [SMILES: CC(C)[C@]1(C(=O)O)CC[C@@H](c2nc(Br)c3c(N)nccn23)C1 ; CC(C)[C@@]1(C(=O)O)CC[C@H](c2nc(Br)c3c(N)nccn23)C1 ; CC(C)[C@@]1(C(=O)O)CC[C@@H](c2nc(Br)c3c(N)nccn23)C1 ; CC(C)[C@]1(C(=O)O)CC[C@H](c2nc(Br)c3c(N)nccn23)C1]

### (1S, 3R) 3-(8-amino-1-bromoimidazo[1,5-a]pyrazin-3-yl)-1-isopropylcyclopentanecarboxlic acid

### (1R, 3S) 3-(8-amino-1-bromoimidazo[1,5-a]pyrazin-3-yl)-1-isopropylcyclopentanecarboxvlic acid

### (1R, 3R) 3-(8-amino-1-bromoimidazo[1,5-a]pyrazin-3-yl)-1-isopropylcyclopentanecarboxvlic acid

### (1S, 3S) 3-(8-amino-1-bromoimidazo[1,5-a]pyrazin-3-yl)-1-isopropylcyclopentanecarboxvlic acid

### Step 1: dimethyl cyclopentane-1, 3-dicarboxylate

To a solution of 3-(methoxycarbonyl)cyclopentanecarboxylic acid (18 g, 104.6 mmol) in MeOH (200 mL) was added H₂SO₄ (30.8 g, 313.9 mmol). The mixture was stirred at 70° C. for 2 h. The reaction was complete detected by TLC. The solvent was concentrated and the residue was purified by column chromatography on silica gel eluted with PE/EA=4/1 to give dimethyl cyclopentane-1,3-dicarboxylate. ¹H NMR (CDCl₃, 400 MHz) δ 1.85-2.01 (m, 4H), 2.04-2.12 (m, 1H), 2.18-2.26 (m, 1H), 2.69-2.87 (m, 2H), 3.66 (s, 6H).

### Step 2: dimethyl 1-isopropylcyclopentane-1,3-dicarboxylate

To a solution of LDA (14.8 mL, 29.6 mmol) in THF (80 mL) and HMPT (19.2 g, 107.5 mmol) at −65° C. was added dropwise a solution of dimethyl cyclopentane-1,3-dicarboxylate (5 g, 26.9 mmol) in THF (20 mL). The mixture was stirred at −65° C. for 10 min, 2-iodopropane (9.1 g, 53.8 mmol) was then added dropwise and the resulting mixture was stirred at room temperature for 4 h. The reaction mixture was quenched with saturated NH₄Cl solution and extracted with EA. The organic layer was dried and concentrated, the residue was purified by column chromatography on silica gel eluted with PE/EA=10/1 to give dimethyl 1-isopropylcyclo pentane-1,3-dicarboxylate. ¹H NMR (CDCl₃, 400 MHz) δ 0.78-0.95 (m, 6H), 1.47-1.79 (m, 2H), 1.82-2.00 (m, 3H), 2.11-2.30 (m, 1H), 2.35-2.52 (m, 1H), 2.69-2.85 (m, 1H), 3.59-3.70 (m, 6H).

### Step 3: 3-isopropyl-3-(methoxycarbonyl)cyclopentanecarboxylic acid

To a solution of dimethyl 1-isopropylcyclopentane-1,3-dicarboxylate (5.4 g, 23.7 mmol) in THF/MeOH/H₂O (50 mL/50 mL/20 mL) was added LiOH.H₂O (3 g, 71.1 mmol). The mixture was stirred at room temperature for 4 h. The reaction mixture was diluted with water, acidified by 1 M HCl, and extracted with EA. The organic layer was dried and concentrated. The residue was used in next step directly.

### Step 4: methyl 3-(((3-chloropyrazin-2-yl)methyl)carbamoyl)-1-isopropylcyclopentane carboxylate

To a solution of 3-isopropyl-3-(methoxycarbonyl)cyclopentanecarboxylic acid (5.1 g, 23.8 mmol) in THF (130 mL) was added (3-chloropyrazin-2-yl)methanamine (6.4 g, 35.7 mmol), HATU (13.6 g, 35.7 mmol) and TEA (14.4 g, 143 mmol). The mixture was stirred at room temperature overnight. The reaction mixture was treated with EA and water, the organic layer was dried and concentrated. The residue was purified by column chromatography on silica gel eluted with PE/THF=10/1 to give methyl 3-(((3-chloropyrazin-2-yl)methyl)carbamoyl)-1-isopropylcyclopentanecarboxylate. ¹H NMR (CDCl₃, 400 MHz) δ 0.89-0.94 (m, 6H), 1.62-1.82 (m, 2H), 1.91-2.29 (m, 4H), 2.43-2.55 (m, 1H), 2.77-2.93 (m, 1H), 3.70 (d, J=3.52 Hz, 3H), 4.65-4.78 (m, 2H), 8.33-8.39 (m, 1H), 8.45-8.52 (m, 1H).

### Step 5: methyl 3-(8-chloroimidazo[1,5-a]pyrazin-3-yl)-1-isopropylcyclopentanecarboxylate

To a solution of methyl 3-(((3-chloropyrazin-2-yl)methyl)carbamoyl)-1-isopropylcyclopentane carboxylate (2 g, 5.9 mmol) in MeCN (100 mL) was added PCl₅ (2.45 g, 11.8 mmol). The mixture was stirred at 65° C. for 2 h. The reaction solution was treated with DCM and aq. NaHCO₃. The organic layer was dried and concentrated. The residue was purified by column chromatography on silica gel eluted with DCM/THF=50/1 to give methyl 3-(8-chloroimidazo[1,5-a]pyrazin-3-yl)-1-isopropylcyclo pentanecarboxylate. MS-ESI (m/z): 322 [M+1]⁺

### Step 6: methyl 3-(1-bromo-8-chloroimidazo[1,5-a]pyrazin-3-yl)-1-isopropylcyclo pentanecarboxylate

To a solution of methyl 3-(8-chloroimidazo[1,5-a]pyrazin-3-yl)-1-isopropylcyclopentanecarboxylate (1.7 g, 5.3 mmol) in DMF (30 mL) was added a solution of NBS (1 g, 5.8 mmol) in DMF (10 mL). The mixture was stirred at room temperature for 1 h. The reaction mixture was treated with EA and water, the organic layer was dried and concentrated. The residue was purified by column chromatography on silica gel eluted with DCM/THF=50/1 to give methyl 3-(1-bromo-8-chloroimidazo[1,5-a]pyrazin-3-yl)-1-isopropylcyclopentane carboxylate. MS-ESI (m/z): 402 [M+1]⁺

### Step 7: methyl 3-(1-bromo-8-((2,4-dimethoxybenzyl)amino)imidazo[1,5-a]pyrazin-3-yl)-1-isopropyl cyclopentanecarboxylate

To a solution of methyl 3-(1-bromo-8-chloroimidazo[1,5-a]pyrazin-3-yl)-1-isopropylcyclo pentanecarboxylate (2 g, 5 mmol) in DMF (40 mL) was added K₂CO₃ (1.38 g, 10 mmol) and (2,4-dimethoxyphenyl)methanamine (921 mg, 5.5 mmol). The mixture was stirred at 90° C. for 6 h. The reaction was treated with EA and water. The organic layer was dried and concentrated. The residue was purified by column chromatography on silica gel eluted with PE/DCM/EA=1/1/0.2 to give methyl 3-(1-bromo-8-((2,4-dimethoxy benzyl)amino)imidazo[1,5-a]pyrazin-3-yl)-1-isopropylcyclopentanecarboxylate. ¹H NMR (CDCl₃, 400 MHz) δ 0.74-0.93 (m, 6H), 1.10-1.28 (m, 1H), 1.52-1.64 (m, 1H), 1.73-1.83 (m, 1H), 1.97-2.10 (m, 3H), 2.33-2.47 (m, 1H), 3.15-3.28 (m, 1H), 3.55-3.69 (m, 3H), 3.73 (s, 3H), 3.81 (s, 3H), 4.59 (d, J=5.6 Hz, 2H), 6.34-6.47 (m, 2H), 6.59-6.68 (m, 1H), 6.94-7.03 (m, 2H), 7.16-7.23 (m, 1H) ppm.

### Step 8: 3-(1-bromo-8-((2,4-dimethoxybenzyl)amino)imidazo[1,5-a]pyrazin-3-yl)-1-isopropylcyclopentanecarboxylic acid

To a solution of methyl 3-(1-bromo-8-((2,4-dimethoxybenzyl)amino)imidazo[1,5-a]pyrazin-3-yl)-1-isopropylcyclopentanecarboxylate (1.2 g, 2.26 mmol) in MeOH (40 mL) was added KOH (1.6 g, 29.4 mmol) and 18-Crown-6 (200 mg). The mixture was stirred at 80° C. overnight. Then the mixture was added THF/H₂O (20 mL/10 mL), and stirred at 80° C. for further 3 days. The reaction was neutralized with 2 M HCl and extracted with EA. The organic layer was dried and concentrated. The residue was purified by column chromatography on silica gel eluted with DCM/EA=2/1 to give 3-(1-bromo-8-((2,4-dimethoxybenzyl)amino)imidazo[1,5-a]pyrazin-3-yl)-1-isopropylcyclopentanecarboxylic acid. MS-ESI (m/z): 517 [M+1]⁺.

### Step 9: 3-(8-amino-1-bromoimidazo[1,5-a]pyrazin-3-yl)-1-isopropylcyclopentanecarboxvlic acid

A solution of 3-(1-bromo-8-((2,4-dimethoxybenzyl)amino)imidazo[1,5-a]pyrazin-3-yl)-1-isopropylcyclopentanecarboxylic acid (1 g, 1.9 mmol) in TFA (6 mL) was heated to reflux for 2 h. The mixture was concentrated and the residue was purified by pre-HPLC to give cis-3-(8-amino-1-bromoimidazo[1,5-a]pyrazin-3-yl)-1-isopropylcyclopentanecarboxylic acid (350 mg), and trans-3-(8-amino-1-bromoimidazo[1,5-a]pyrazin-3-yl)-1-isopropylcyclopentanecarboxylic acid (300 mg), which were separated with SFC to give (1S,3R)-3-(8-amino-1-bromoimidazo[1,5-a]pyrazin-3-yl)-1-isopropylcyclopentanecarboxylic acid (100 mg), (1R,3S)-3-(8-amino-1-bromoimidazo[1,5-a]pyrazin-3-yl)-1-isopropylcyclopentanecarboxylic acid (90 mg), (1R,3R)-3-(8-amino-1-bromoimidazo[1,5-a]pyrazin-3-yl)-1-isopropylcyclopentanecarboxylic acid (90 mg) and (1S,3S)-3-(8-amino-1-bromoimidazo[1,5-a]pyrazin-3-yl)-1-isopropylcyclopentanecarboxylic acid. SFC condition: For cis mixture: “Column: Chiralpak AD-H 250×4.6 mm I.D., 5 um Mobile phase: ethanol (0.05% DEA) in CO₂ from 5% to 40% Flow rate: 2.35 mL/min Wavelength: 220 nm”.

¹H NMR (CD₃OD, 400 MHz) δ 0.97 (dd, J=11.17, 6.90 Hz, 6H), 1.69-1.82 (m, 1H), 2.01-2.23 (m, 4H), 2.40-2.59 (m, 2H), 3.48-3.61 (m, 1H), 6.92 (d, J=6.0 Hz, 1H), 7.71 (d, J=6.0 Hz, 1H).

For trans mixture: “Column: Chiralpak AD-H 250×4.6 mm I.D., 5 um Mobile phase: ethanol (0.05% DEA) in CO₂ from 5% to 40% Flow rate: 2.35 mL/min Wavelength: 220 nm”.

¹H NMR (CD₃OD, 400 MHz) δ 0.98 (dd, J=10.92, 6.90 Hz, 6H), 1.88-1.98 (m, 3H), 2.04-2.34 (m, 3H), 2.60-2.69 (m, 1H), 3.52-3.62 (m, 1H), 6.91 (d, J=6.0 Hz, 1H), 7.68 (d, J=6.0 Hz, 1H).

### Intermediate 8

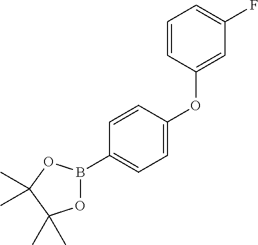 [SMILES: CC1(C)OB(c2ccc(Oc3cccc(F)c3)cc2)OC1(C)C]

### 2-(4-(3-fluorophenoxy)phenyl)-4,4,5,5-tetramethyl-1,3,2-dioxaborolane

### Step 1: 1-(4-bromophenoxy)-3-fluorobenzene

To a solution of (3-fluorophenyl)boronic acid (4360 mg, 31.161 mmol) in dry DCM (60 mL) was added 4-bromophenol (5013 mg, 28.979 mmol), Cu(OAc)₂ (6226 mg, 34.277 mmol) and TEA (6306 mg, 62.321 mmol), and the resulting mixture was stirred at room temperature for 12 h. The mixture was extracted with DCM three times. The combined organic extracts were washed with brine, dried over anhydrous sodium sulfate, filtered and concentrated in vacuum. The residue was purified by column chromatography on silica gel (PE/EA=20/1) to afford the 1-(4-bromophenoxy)-3-fluorobenzene.

### Step 2: 2-(4-(3-fluorophenoxy)phenyl)-4,4,5,5-tetramethyl-1,3,2-dioxaborolane

To a solution of 1-(4-bromophenoxy)-3-fluorobenzene (800 mg, 2.996 mmol) in dry dioxane (10 mL) was added 4,4,4′,4′,5,5,5′,5′-octamethyl-2,2′-bi(1,3,2-dioxaborolane) (1142 mg, 4.494 mmol), KOAc (882 mg, 8.988 mmol) and Pd(dppf)Cl₂ (110 mg, 0.150 mmol) under nitrogen protection, and the mixture was stirred at 90° C. for 12 h. Concentrate in vacuum to remove the solvent, then the mixture was extracted with ethyl acetate three times. The combined organic extracts were washed with brine, dried over anhydrous sodium sulfate, filtered and concentrated in vacuum. The residue was purified by column chromatography on silica gel (PE/EA=20/1) to afford 2-(4-(3-fluorophenoxy)phenyl)-4,4,5,5-tetramethyl-1,3,2-dioxaborolane. ¹H NMR (CDCl₃, 400 MHz) δ 1.34 (s, 12H), 6.71 (dd, J=9.98, 2.15 Hz, 1H), 6.76-6.83 (m, 2H), 6.98-7.02 (m, 2H), 7.23-7.29 (m, 1H), 7.80 (m, J=8.61 Hz, 2H) ppm.

### Intermediate 9

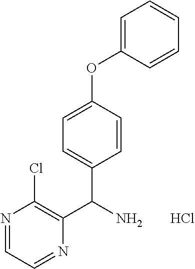 [SMILES: NC(c1ccc(Oc2ccccc2)cc1)c1nccnc1Cl ; Cl]

### (3-chloropyrazin-2-yl)(4-phenoxyphenyl)methanamine hydrochloride

### Step 1: (E)-N-((3-chloropyrazin-2-yl)methylene)-2-methylpropane-2-sulfinamide

To a solution of 3-chloropyrazine-2-carbaldehyde (900 mg, 6.31 mmol) and 2-methylpropane-2-sulfinamide (842 mg, 6.95 mmol) in methylene chloride (10 mL), was added cupric sulfate (1.109 g, 6.95 mmol). The mixture was stirred at room temperature for 17 h overnight. The mixture was diluted with ethylacetate and washed with water, brine, dried over MgSO₄, filtered and concentrated. The crude was purified by column chromatography (24 g silica gel, 50% ethyl acetacetate in hexanes) to afford product (E)-N-((3-chloropyrazin-2-yl)methylene)-2-methylpropane-2-sulfinamide.

### Step 2: N-((3-chloropyrazin-2-yl)(4-phenoxyphenyl)methyl)-2-methylpropane-2-sulfinamide

To a solution of 1-bromo-4-phenoxybenzene (11.71 g, 47 mol) in THF (110 mL) cooled to −78° C., was added dropwise a solution of n-butyl lithium (2.5 M, 18.8 mL) via syringe over 5 min. Then a solution of (E)-N-((3-chloropyrazin-2-yl)methylene)-2-methylpropane-2-sulfinamide (10.5 g, 47 mmol) in THF (100 mL) was added via syringe over 5 min. The resulting reaction mixture was then stirred at −78° C. for 30 min. The reaction mixture was quenched by addition of NH₄Cl (sat), and then extracted with ethyl acetate (3×), washed with brine, dried over Na₂SO₄, filtered and concentrated. The crude was purified by MPLC (120 g silica gel, 70% ethyl acetate in hexanes) to afford tite compound N-((3-chloropyrazin-2-yl)(4-phenoxyphenyl)methyl)-2-methylpropane-2-sulfinamide. ¹HNMR (CDCl₃, 400 MHz) δ: 8.56 (1H, d), 8.36 (1H, d), 7.45 (2H, t), 7.37 (2H, m), 7.16 (1H, t), 7.06 (2H, d), 6.96 (2H, 2), 5.98 (1H, d), 5.30 (1H, d), 1.24 (9H, s) ppm.

### Step 3: (3-chloropyrazin-2-yl)(4-phenoxyphenyl)methanamine hydrochloride

To a solution of N-((3-chloropyrazin-2-yl)(4-phenoxyphenyl)methyl)-2-methylpropane-2-sulfinamide (17.7 g, 42.7 mmol) in ethyl acetate (400 mL) was added a solution of HCl in dioxane (4M, 22.4 mL). The resulting reaction mixture was then stirred at room temperature for 30 min. To the thick mixture was added heptane (50 mL) and aged for 10 min. The crystalline of the title product (3-chloropyrazin-2-yl)(4-phenoxyphenyl)methanamine hydrochloride was collected and washed with heptane.

### Intermediate 10

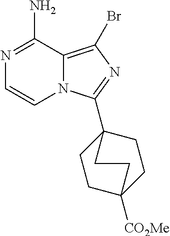 [SMILES: COC(=O)C12CCC(c3nc(Br)c4c(N)nccn34)(CC1)CC2]

### methyl 4-(8-amino-1-bromoimidazo[1,5-a]pyrazin-3-yl)bicyclo[2.2.2]octane-1-carboxylate

### Step 1: methyl 4-(((3-chloropyrazin-2-yl)methyl)carbamoyl)bicyclo[2.2.2]octane-1-carboxylate

A mixture of (3-Chloropyrazin-2-yl)methanamine dihydrochloride (22.95 g, 106 mmol), 4-(methoxycarbonyl)bicyclo[2.2.2]octane-1-carboxylic acid (15 g, 70.7 mmol), HATU (32.2 g, 85 mmol) and TEA (59.1 ml, 424 mmol) was stirred in DMF (300 ml) for 2 h. The crude was concentrated to dryness, and the residue was loaded onto column and eluted with 50% EA/Hex to give methyl 4-(((3-chloropyrazin-2-yl)methyl)carbamoyl)bicyclo[2.2.2]octane-1-carboxylate. LCMS Data: m/z 338.07 [M+H]⁺.

### Step 2: methyl 4-(8-chloroimidazo[1,5-a]pyrazin-3-yl)bicyclo[2.2.2]octane-1-carboxylate

Phosphoryl trichloride (5.30 ml, 56.8 mmol) was added to a stirred mixture of methyl 4-(((3-chloropyrazin-2-yl)methyl)carbamoyl)bicyclo[2.2.2]octane-1-carboxylate (6.4 g, 18.95 mmol) in acetonitrile (50 ml) and DMF (2 ml, 25.8 mmol) and the mixture was stirred at 75° C. for 1.5 h. The reaction mixture was concentrated and co-evaporated with dioxane to give methyl 4-(8-chloroimidazo[1,5-a]pyrazin-3-yl)bicyclo[2.2.2]octane-1-carboxylate as an oil, which was used in the next step without further purification. LCMS Data: m/z 320.13 [M+H]⁺.

### Step 3: methyl 4-(1-bromo-8-chloroimidazo[1,5-a]pyrazin-3-yl)bicyclo[2.2.2]octane-1-carboxylate

NBS (3.40 g, 19.08 mmol) was added to the methyl 4-(8-chloroimidazo[1,5-a]pyrazin-3-yl)bicyclo[2.2.2]octane-1-carboxylate (0347906-0005-cd) (6.1 g, 19.08 mmol) in DMF (20 ml) and the reaction was stirred at room temperature for 2 h. LCMS showed M+H at 400 and SM, another 1 g of NBS was added and the resulting mixture was stirred for overnight, diluted with EA and treated with sat. sodium bicarbonate, washed with water, brine and dried and concentrated. The residue was purified on column with 30% ethyl acetate in hexanes to give methyl 4-(1-bromo-8-chloroimidazo[1,5-a]pyrazin-3-yl)bicyclo[2.2.2]octane-1-carboxylate. LCMS Data: m/z 400.02 [M+H]⁺.

### Step 4: methyl 4-(8-amino-1-bromoimidazo[1,5-a]pyrazin-3-yl)bicyclo[2.2.2]octane-1-carboxylate

A stirred mixture of methyl 4-(1-bromo-8-chloroimidazo[1,5-a]pyrazin-3-yl)bicyclo[2.2.2]octane-1-carboxylate (Ig, 2.508 mmol) in solution of ammonia (60 ml, 120 mmol) (2N in 2-propanol) was stirred at 120° C. in a sealed tube for overnight. After cooled to room temperature, the mixture was concentrated. The residue was purified by column chromatography on silica gel eluting with CH₂Cl₂/MeOH (50/1) to give methyl 4-(8-amino-1-bromoimidazo[1,5-a]pyrazin-3-yl)bicyclo[2.2.2]octane-1-carboxylate as a solid. LCMS Data: m/z 381.02 [M+H]⁺.

### Intermediate 11

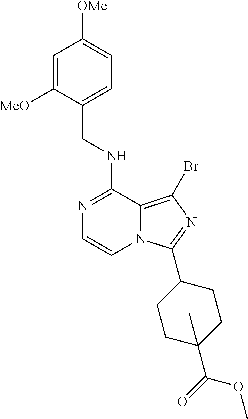

### Trans-methyl 4-(1-bromo-8-((2,4-dimethoxybenzyl)amino)imidazo[1,5-a]pyrazin-3-yl)-1-methylcyclohexanecarboxylate

### Step 1: dimethyl 1-methylcyclohexane-1,4-dicarboxylate

A solution of LDA (2 N, 30.0 ml, 59.9 mmol) was added to a mixture of dimethyl cyclohexane-1,4-dicarboxylate (10 g, 49.9 mmol) and 1,3-dimethyl-3,4,5,6-tetrahydro-2(1H)-pyridineone (27.1 ml, 225 mmol) in THF (4 ml) at −78° C. and the reaction mixture was stirred at −78° C. for 30 min. The iodomethane (3.11 ml, 49.9 mmol) was added and stirred and allowed slowly warm up from −78° C. to rt and stirred at rt for 3 h. The reaction mixture was treated with sat. NH₄Cl and extracted with ethyl acetate, dried and concentrated. The crude was purified on column with 10% EA/hexanes to give dimethyl 1-methylcyclohexane-1,4-dicarboxylate.

### Step 2: 4-(methoxycarbonyl)-4-methylcyclohexanecarboxylic acid

A solution of LiOH (2N, 27.9 ml, 55.9 mmol) was added to a mixture of dimethyl 1-methylcyclohexane-1,4-dicarboxylate (9.98 g, 46.6 mmol) in MeOH (50 ml) and THF (50 ml), the reaction was stirred at 40° C. for 30 min. The reaction mixture was neutralized with 1N HCl and concentrated to dryness to give a crude, 4-(methoxycarbonyl)-4-methylcyclohexanecarboxylic acid, which was used as is for next step without further purification.

### Step 3: methyl 4-(((3-chloropyrazin-2-yl)methyl)carbamoyl)-1-methylcyclohexanecarboxylate

A mixture of (3-chloropyrazin-2-yl)methanamine dihydrochloride (10 g, 46.6 mmol), 4-(methoxycarbonyl)-4-methylcyclohexanecarboxylic acid (9.33 g, 46.4 mmol), HATU (19.5 g, 51.3 mmol) and DIPEA (48.4 mL, 280 mmol) was stirred in DCM (100 mL) for 2 h. The reaction mixture was concentrated and the residue was purified on column, eluting with EtOAc/isohexane (1/1) to give methyl 4-(((3-chloropyrazin-2-yl)methyl)carbamoyl)-1-methylcyclohexanecarboxylate. LCMS Data: m/z 326.09 [M+H]⁺.

### Step 4: methyl 4-(8-chloroimidazo[1,5-a]pyrazin-3-yl)-1-methylcyclohexanecarboxylate

POCl₃ (7.57 ml, 81 mmol) was added to a stirred mixture of methyl 4-(((3-chloropyrazin-2-yl)methyl)carbamoyl)-1-methylcyclohexanecarboxylate (7.56 g, 23.21 mmol) in acetonitrile (100 ml) and stirred at 70° C. for 45 min. The reaction mixture was concentrated and azotroped with dioxane to give methyl 4-(8-chloroimidazo[1,5-a]pyrazin-3-yl)-1-methylcyclohexanecarboxylate as syrup, which was used in next step without further purification. LCMS Data: m/z 308.08 [M+H]⁺.

### Step 5: methyl 4-(1-bromo-8-chloroimidazo[1,5-a]pyrazin-3-yl)-1-methylcyclohexanecarboxylate

NBS (4.13 g, 23.20 mmol) was added to a mixture of methyl 4-(8-chloroimidazo[1,5-a]pyrazin-3-yl)-1-methylcyclohexanecarboxylate (7.14 g, 23.20 mmol) in DMF (100 ml). The reaction mixture was stirred for overnight and treated with Sat. sodium bicarbonate, stirred for 15 min, extracted with ethyl acetate, dried and concentrated. The residue was purified on column with 50% EA in hexanes to give methyl 4-(1-bromo-8-chloroimidazo[1,5-a]pyrazin-3-yl)-1-methylcyclohexanecarboxylate. LCMS Data: m/z 387.78 [M+H]⁺.

### Step 6: methyl 4-(1-bromo-8-((2,4-dimethoxybenzyl)amino)imidazo[1,5-a]pyrazin-3-yl)-1-methylcyclohexanecarboxylate

To a stirred mixture of methyl 4-(1-bromo-8-chloroimidazo[1,5-a]pyrazin-3-yl)-1-methylcyclohexanecarboxylate (0.46 g, 1.19 mmol) and DIPEA (0.721 mL, 4.16 mmol) in 1,4-dioxane (100 ml) was added (2,4-dimethoxyphenyl)methanamine (0.63 mL, 4.16 mmol) and the mixture was stirred at room temperature for overnight. More DMB amine (2 eq) was added and stirred for another 4 h. LCMS showed 70% conversion. More DMB amine (2 eq) was added and the resulting mixture was stirred for overnight, washed with 5% KH₂PO₄ and extracted with EA, dired and concentrated. The residue was purified on column with 5% MeOH in DCM to give methyl 4-(1-bromo-8-((2,4-dimethoxybenzyl)amino)imidazo[1,5-a]pyrazin-3-yl)-1-methylcyclohexanecarboxylate. LCMS Data: m/z 519.11 [M+H]⁺.

### Intermediate 12

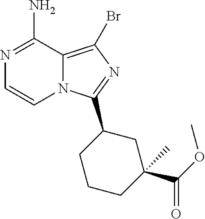 [SMILES: COC(=O)[C@@]1(C)CCC[C@@H](c2nc(Br)c3c(N)nccn23)C1]

### (1S,3R)-3-(8-amino-1-bromoimidazo[1,5-a]pyrazin-3-yl)-1-methylcyclohexanecarboxylic acid

### Step 1: cis-dimethyl 1-methylcyclohexane-1,3-dicarboxylate

A solution of LDA (5.99 ml, 11.99 mmol) was added to a mixture of dimethyl cyclohexane-1,3-dicarboxylate (2 g, 9.99 mmol) and 1,3-dimethyl-3,4,5,6-tetrahydro-2(1H)-pyridineone (5.42 ml, 44.9 mmol) in THF (4 ml) at −78° C. and the reaction mixture was stirred at −78° C. for 30 min. Iodomethane (0.622 ml, 9.99 mmol) was added, the mixture was stirred and allowed slowly warm up from −78° C. to room temperature and stirred further at rt for 4 h. The reaction mixture was treated with sat. NH₄Cl and extracted with ethyl acetate, dried and concentrated. The residue was purified by column with 10% ethyl acetate in hexanes to give cis-dimethyl 1-methylcyclohexane-1,3-dicarboxylate.

### Step 2: cis-3-(methoxycarbonyl)-3-methylcyclohexanecarboxylic acid

LiOH (1M aqueous) (26.6 ml, 26.6 mmol) was added to a mixture of cis-dimethyl 1-methylcyclohexane-1,3-dicarboxylate (1.9 g, 8.87 mmol) in MeOH (20 ml) and THF (20 ml), the reaction mixture was stirred at 40° C. for 30 min. The reaction mixture was treated with 1.5 eq of 1N HCl, extracted with ethyl acetate, dried and concentrated to give cis-3-(methoxycarbonyl)-3-methylcyclohexanecarboxylic acid.

### Step 3: cis-methyl 3-(((3-chloropyrazin-2-yl)methyl)carbamoyl)-1-methylcyclohexane carboxylate

HATU (4.33 g, 11.39 mmol) was added to a stirred mixture of (3-chloropyrazin-2-yl)methanamine hydrochloride (2.050 g, 11.39 mmol), 3-(methoxycarbonyl)-3-methylcyclohexanecarboxylic acid (1.9 g, 9.49 mmol) and DIPEA (4.97 ml, 28.5 mmol) in DCM (100 ml) and the mixture was stirred at room temperature for 2 h. The mixture was then concentrated and the residue was loaded onto cartridge (Isco, 120 g), eluted with EtOAc/isohexane (1/1) to give cis-methyl 3-(((3-chloropyrazin-2-yl)methyl)carbamoyl)-1-methylcyclohexanecarboxylate (3 g, 9.21 mmol, 97% yield). LCMS Data: m/z 326.09 [M+H]⁺.

### Step 4: cis-methyl 3-(8-chloroimidazo[1,5-a]pyrazin-3-yl)-1-methylcyclohexanecarboxylate

POCl₃ (3.06 ml, 32.8 mmol) was added to a stirred mixture of methyl 3-(((3-chloropyrazin-2-yl)methyl)carbamoyl)-1-methylcyclohexanecarboxylate (2.67 g, 8.20 mmol) in acetonitrile (2 ml) and stirred at 70° C. for 45 min. The reaction mixture was then concentrated down to give cis-methyl 3-(8-chloroimidazo[1,5-a]pyrazin-3-yl)-1-methylcyclohexanecarboxylate as syrup, which is used in next step without further purification. LCMS Data: m/z 308.08 [M+H]⁺.

### Step 5: cis-methyl 3-(1-bromo-8-chloroimidazo[1,5-a]pyrazin-3-yl)-1-methylcyclohexanecarboxylate

NBS (1.698 g, 9.54 mmol) was added to a stirred mixture of methyl 3-(8-chloroimidazo[1,5-a]pyrazin-3-yl)-1-methylcyclohexanecarboxylate (2.67 g, 8.68 mmol) in DMF (60 ml) and the mixture was stirred at room temperature for 1 h. The mixture was then diluted with EtOAc, washed with sat. NaHCO₃, water, dried over Na₂SO₄, filtered and concentrated. The resultant crude was purified on column to give cis-methyl 3-(1-bromo-8-chloroimidazo[1,5-a]pyrazin-3-yl)-1-methylcyclohexanecarboxylate (2 g). LCMS Data: m/z 387.93 [M+H]⁺.

### Step 6: cis-methyl 3-(1-bromo-8-((2,4-dimethoxybenzyl)amino)imidazo[1,5-a]pyrazin-3-yl)-1-methylcyclohexanecarboxylate

A mixture of (2,4-dimethoxyphenyl)methanamine (2.75 mL, 18.1 mmol), cis-methyl 3-(1-bromo-8-chloroimidazo[1,5-a]pyrazin-3-yl)-1-methylcyclohexanecarboxylate (2 g, 5.17 mmol) and N-ethyl-N-isopropylpropan-2-amine (3.14 mL, 18.1 mmol) was stirred in 1,4-Dioxane (100 ml) for overnight.). More (2,4-dimethoxyphenyl)methanamine (2 eq) was added and stirred for another 4 h. More DMB amine (2 eq) was added and stirred for overnight. The reaction mixture was washed with 5% KH2PO4 and extracted with ethyl acetate (3×), dried and concentrated. The residue was purified on column with 5% ethyl acetae in hexanes to give cis-methyl 3-(1-bromo-8-((2,4-dimethoxybenzyl)amino)imidazo[1,5-a]pyrazin-3-yl)-1-methylcyclohexanecarboxylate. LCMS Data: m/z 509.06 [M+H]⁺.

### Step 7: (1S,3R)-methyl 3-(1-bromo-8-((2,4-dimethoxybenzyl)amino)imidazo[1,5-a]pyrazin-3-yl)-1-methylcyclohexanecarboxylate and (1R,3S)-methyl 3-(1-bromo-8-((2,4-dimethoxybenzyl)amino)imidazo[1,5-a]pyrazin-3-yl)-1-methylcyclohexanecarboxylate

Cis-Methyl 3-(1-bromo-8-((2,4-dimethoxybenzyl)amino)imidazo[1,5-a]pyrazin-3-yl)-1-methylcyclohexanecarboxylate (1.2 g, 2.319 mmol) was purified by SFC-HPLC on a Chiralpak AD, 30×250 mm, 35% 2:1 MeOH/MeCN, 60 ml/min, ˜70 mg/ml in 2:1 MeCN/MeOH into two isomers, (1 S,3R)-methyl 3-(1-bromo-8-((2,4-dimethoxybenzyl)amino)imidazo[1,5-a]pyrazin-3-yl)-1-methylcyclohexanecarboxylate (351.4 mg, 0.679 mmol, 29.3% yield) and (1R,3S)-methyl 3-(1-bromo-8-((2,4-dimethoxybenzyl)amino)imidazo[1,5-a]pyrazin-3-yl)-1-methylcyclohexanecarboxylate.

### Step 8: (1S,3R)-3-(1-bromo-8-((2,4-dimethoxybenzyl)amino)imidazo[1,5-a]pyrazin-3-yl)-1-methylcyclohexanecarboxylic acid

LiOH (3.5 mL, 3.50 mmol) was added to the mixture of (1S,3R)-methyl 3-(1-bromo-8-((2,4-dimethoxybenzyl)amino)imidazo[1,5-a]pyrazin-3-yl)-1-methylcyclohexanecarboxylate (350 mg, 0.676 mmol) in THF (5 ml)/MeOH (5 ml) at 60° C. overnight. Another 5 eq of LiOH was added and stirred at 80° C. for 4 h.

The mixture was concentrated by Rotovap to dryness to give (1S,3R)-3-(1-bromo-8-((2,4-dimethoxybenzyl)amino)imidazo[1,5-a]pyrazin-3-yl)-1-methylcyclohexanecarboxylic acid, which was used as is for next reaction. LCMS Data: m/z 504.93 [M+H]⁺.

### Step 9: (1S,3R)-3-(8-amino-1-bromoimidazo[1,5-a]pyrazin-3-yl)-1-methylcyclohexane carboxylic acid

Triethylsilane (0.055 ml, 0.342 mmol) was added to a mixture of (1S,3R)-3-(1-bromo-8-((2,4-dimethoxybenzyl)amino)imidazo[1,5-a]pyrazin-3-yl)-1-methylcyclohexanecarboxylic acid (172 mg, 0.342 mmol) in TFA (5 ml) and the r×n was stirred at 75° C. for 2 h and concentrated. The residue was purified on column with 25% MeOH in DMC to give (1S,3R)-3-(8-amino-1-bromoimidazo[1,5-a]pyrazin-3-yl)-1-methylcyclohexanecarboxylic acid. LCMS Data: m/z 355.02 [M+H]⁺.

### Intermediate 13

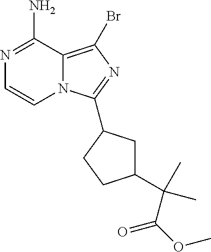 [SMILES: COC(=O)C(C)(C)C1CCC(c2nc(Br)c3c(N)nccn23)C1]

### Methyl 2-(3-(8-amino-1-bromoimidazo[1,5-a]pyrazin-3-yl)cyclopentyl)-2-methylpropanoate

### Step 1: methyl 2-methyl-2-(3-oxocyclopentyl)propanoate

To a solution of lithium acetate (0.057 g, 0.861 mmol) in DMF (30 mL) was successively added cyclopent-2-enone (0.989 g, 12.05 mmol) and ((1-methoxy-2-methylprop-1-en-1-yl)oxy)trimethylsilane (1.5 g, 8.61 mmol). The solution was stirred at RT. To the solution was added 30 mL 1N HCl and stirred for another 1 hour and extracted with EtOAc. The combined organic was washed with water and brine, dried over Na₂SO₄, concentrated in vacuo and purified by chromatography over silica gel to give the title compound. ¹H NMR (CDCl₃ 400 MHz): δ 3.69 (s, 3H), 2.53-2.37 (m, 1H), 2.39-1.98 (m, 6H), 1.72-1.58 (m, 1H), 1.21 (d, J=3.5 Hz, 6H) ppm.

### Step 2: methyl 2-(3-formylcyclopentyl)-2-methylpropanoate

To a solution of (Methoxymethyl)triphenylphosphonium chloride (2791 mg, 8.14 mmol) in THF (30 mL) was added s-BuLi (5.64 mL, 7.33 mmol) at 0° C. under N₂ protection. The solution was stirred at 0° C. for 1 hour. To the solution was added a solution of methyl 2-methyl-2-(3-oxocyclopentyl)propanoate (750 mg, 4.07 mmol) in 10 mL THF dropwise and stirred at room temperature for 16 hours. The solution was added 3N HCl to pH=1 and stirred for another 1 hour. The solution was extracted with EtOAc. The combined organic was washed with NaHCO₃, water and brine, dried over Na₂SO₄ and purified by chromatography over silica gel to give the title compound. ¹H NMR (CDCl₃ 400 MHz):δ 9.58 (d, J=3.5 Hz, 1H), 3.63 (s, 3H), 2.87-2.64 (m, 1H), 2.26-2.05 (m, 1H), 2.00-1.48 (m, 5H), 1.43-1.27 (m, 1H), 1.13 (s, 6H) ppm. MS: 232 (M+1).

### Step 3: 3-(1-methoxy-2-methyl-1-oxopropan-2-yl)cyclopentanecarboxylic acid

To a solution of 2-methyl-2-butene (224 mg, 3.19 mmol) and methyl 2-(3-formylcyclopentyl)-2-methylpropanoate (200 mg, 1.009 mmol) in water (10 mL) and t-BuOH (10 mL, 1.009 mmol) was added a mixture of KH₂PO₄ (1236 mg, 9.08 mmol) and NaClO₂ (639 mg, 7.06 mmol) at RT. The solution was stirred for 16 hours and extracted with EtOAc. The combined organic layers were dried over Na₂SO₄ and concentrated in vacuo to give the crude title compound.

### Step 4: methyl 2-(3-(((3-chloropyrazin-2-yl)methyl)carbamoyl)cyclopentyl)-2-methylpropanoate

To a stirred solution of 3-(1-methoxy-2-methyl-1-oxopropan-2-yl)cyclopentane carboxylic acid (214 mg, 0.999 mmol) in DCM (4 mL) was added HATU (456 mg, 1.199 mmol) and Et₃N (0.348 mL, 2.497 mmol). The solution was stirred at room temperature for 30 minutes. Then to this solution was added (3-chloropyrazin-2-yl)methanamine (172 mg, 1.199 mmol). The mixture was stirred at RT overnight. The reaction mixture was purified by chromatography (petroleum ether: ethyl acetate=1:1) to give the crude title compound.

### Step 5: methyl 2-(3-(8-chloroimidazo[1,5-a]pyrazin-3-yl)cyclopentyl)-2-methyl propanoate

A solution of methyl 2-(3-(((3-chloropyrazin-2-yl)methyl)carbamoyl)cyclopentyl)-2-methylpropanoate (712 mg, 2.095 mmol) and PCl₅ (1309 mg, 6.29 mmol) in acetonitrile (10 mL) was stirred at 80° C. for 2 hours and poured into 10% NaHCO₃. The mixture was extracted with EtOAc. The combined organic was dried over K₂CO₃ and concentrated in vacuo. The residue was purified by column chromatography (petroleum ether: ethyl acetate=1:1) to give the title compound.

### Step 6: methyl 2-(3-(1-bromo-8-chloroimidazo[1,5-a]pyrazin-3-yl)cyclopentyl)-2-methylpropanoate

To solution of methyl 2-(3-(8-chloroimidazo[1,5-a]pyrazin-3-yl)cyclopentyl)-2-methylpropanoate (540 mg, 1.678 mmol) in acetonitrile (8 mL) was added 1-bromopyrrolidine-2,5-dione (329 mg, 1.846 mmol), then the reaction mixture was stirred at 20° C. for 1 h. The reaction mixture was concentrated under reduced pressure to give the crude title compound. MS: 402 (M+1).

### Step 7: methyl 2-(3-(8-amino-1-bromoimidazo[1,5-a]pyrazin-3-yl)cyclopentyl)-2-methylpropanoate

A solution of methyl 2-(3-(1-bromo-8-chloroimidazo[1,5-a]pyrazin-3-yl)cyclopentyl)-2-methylpropanoate (350 mg, 0.873 mmol) in ammonium hydroxide (9 mL, 64.7 mmol) was added iPrOH (6 mL, 78 mmol). The reaction mixture was stirred at 100° C. for 16 hours under sealed tube. The reaction mixture was concentrated in vacuo, dissolved in EtOAc, washed with water. The combined organic layers were dried over Na₂SO₄ and concentrated in vacuo to give the title compound. MS: 381/383 (M+1).

### Intermediate 14

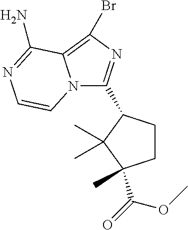 [SMILES: COC(=O)[C@@]1(C)CC[C@@H](c2nc(Br)c3c(N)nccn23)C1(C)C]

### (1S,3R)-methyl 3-(8-amino-1-bromoimidazo[1,5-a]pyrazin-3-yl)-1,2,2-trimethylcyclopentanecarboxylate

### Step 1: (1S,3R)-dimethyl 1, 2,2-trimethylcyclopentane-1,3-dicarboxylate

To a suspension of (1S, 3R)-1,2,2-trimethylcyclopentane-1,3-dicarboxylic acid (4.87 g, 24.32 mmol) and K₂CO₃ (10.08 g, 73.0 mmol) in DMF (50 mL) was stirred at 13° C. and iodomethane (12.62 g, 89 mmol) was added. Then the mixture was stirred at 13° C. for 3 h. The reaction was complete detected by TLC. The reaction mixture was added EtOAc (100 mL) and washed with water and brine, dried over Na₂SO₄ and concentrated to give the title compound. ¹H NMR (400 MHz, MeOH-d4): δ 3.66 (d, J=3.9 Hz, 6H), 2.87 (t, J=9.4 Hz, 1H), 2.54 (dt, J=7.6, 12.6 Hz, 1H), 2.21-2.08 (m, 1H), 1.90-1.76 (m, 1H), 1.50 (ddd, J=3.9, 9.6, 13.5 Hz, 1H), 1.22 (d, J=12.5 Hz, 6H), 0.73 (s, 3H).

### Step 2: (1S,3R)-3-(methoxycarbonyl)-2,2,3-trimethylcyclopentanecarboxylic acid

To a solution of (1S,3R)-dimethyl 1,2,2-trimethylcyclopentane-1,3-dicarboxylate (5.37 g, 23.52 mmol) and lithium hydroxide hydrate (0.987 g, 23.52 mmol) in MeOH (60 mL) was stirred at 10° C. for 40 h. The mixture was concentrated to remove MeOH (20 mL), and then 5 mL of water was added and stirred at 30° C. for overnight. The mixture was heated to 50° C. for overnight. The mixture was concentrated to remove solvent, and then added water (100 mL), washed with EtOAc. The water layer was added HCl to adjust to pH=3, and then the mixture was extracted with EtOAc (3×30 mL), the organic layer was dried over Na₂SO₄ and concentrated to give the title compound. ¹H NMR (400 MHz, CDCl₃): δ 3.69 (s, 3H), 2.84 (t, J=9.4 Hz, 1H), 2.59 (dt, J=7.6, 12.6 Hz, 1H), 2.22-2.12 (m, 1H), 1.90-1.78 (m, 1H), 1.53 (ddd, J=3.9, 9.6, 13.5 Hz, 1H), 1.33-1.26 (m, 3H), 1.22 (s, 3H), 0.85 (s, 3H).

### Step 2: (1S,3R)-methyl 3-(((3-chloropyrazin-2-yl)methyl)carbamoyl)-1,2,2-trimethyl cyclopentanecarboxylate

To a mixture of (1S,3R)-3-(methoxycarbonyl)-2,2,3-trimethylcyclopentanecarboxylic acid (4.93 g, 23.01 mmol) and TEA (9.62 mL, 69.0 mmol) and HATU (13.12 g, 34.5 mmol) in DCM (100 mL) was stirred at 12° C. for 30 min. (3-chloropyrazin-2-yl) methanamine hydrochloride (4.97 g, 27.6 mmol) was added and the mixture was stirred at 12° C. for overnight. The reaction was complete detected by LC-MS. The mixture was added DCM (100 mL) and washed and brine. The organic layer was dried over Na₂SO₄, purified with silica gel to give the title compound. ¹H NMR (400 MHz, CDCl₃): δ 8.46 (d, J=2.3 Hz, 1H), 8.34 (s, 1H), 6.70 (br. s., 1H), 4.81-4.66 (m, 2H), 3.69 (s, 3H), 2.74 (t, J=9.2 Hz, 1H), 2.65 (dt, J=6.8, 12.6 Hz, 1H), 2.34-2.22 (m, 1H), 1.91-1.80 (m, 1H), 1.54 (ddd, J=4.1, 9.6, 13.7 Hz, 1H), 1.32 (s, 3H), 1.25 (s, 3H), 0.81 (s, 3H).

### Step 3: (1S,3R)-methyl 3-(8-chloroimidazo[1,5-a]pyrazin-3-yl)-1, 2,2-trimethylcyclo pentanecarboxylate

(1S,3R)-methyl 3-(((3-chloropyrazin-2-yl)methyl)carbamoyl)-1,2,2-trimethylcyclo pentanecarboxylate (7.76 g, 22.84 mmol) was dissolved in acetonitrile (100 mL) and cooled to 0° C., PCl₅ (14.27 g, 68.5 mmol) was added slowly. The mixture was stirred at room temperature for 30 min. The reaction was complete detected by TLC and poured into saturated sodium bicarbonate at 0° C. Then the mixture was extracted with EtOAc, dried over anhydrous sodium sulfate and sodium carbonate, filtered and concentrated to give crude title compound.

### Step 4: (1S,3R)-methyl 3-(1-bromo-8-chloroimidazo[1,5-a]pyrazin-3-yl)-1,2,2-trimethylcyclopentanecarboxylate

NBS (4.28 g, 24.03 mmol) was added in portions to a stirred solution of (1S,3R)-methyl 3-(8-chloroimidazo[1,5-a]pyrazin-3-yl)-1,2,2-trimethylcyclopentane carboxylate (7.03 g, 21.85 mmol) in acetonitrile (80 mL). The mixture was stirred at 12° C. for 1 h. The reaction was added saturated sodium sulfite and extracted with EtOAc, the organic layer was dried over anhydrous sodium sulfate, filtered, concentrated and purified with silica gel to give the title compound. ¹H NMR (400 MHz, CDCl₃): δ 7.69 (d, J=4.7 Hz, 1H), 7.29 (d, J=5.1 Hz, 1H), 3.70 (s, 3H), 3.53 (t, J=9.4 Hz, 1H), 2.82 (dt, J=6.3, 12.9 Hz, 1H), 2.70-2.59 (m, 1H), 2.15-2.06 (m, 1H), 1.67 (ddd, J=4.3, 9.5, 13.6 Hz, 1H), 1.38 (s, 2H), 1.13 (s, 2H), 0.75 (s, 2H) ppm.

### Step 5: (1S,3R)-methyl 3-(8-amino-1-bromoimidazo[1,5-a]pyrazin-3-1)-1,2,2-trimethylcyclopentanecarboxylate

To a mixture of (1S, 3R)-methyl 3-(1-bromo-8-chloroimidazo[1,5-a]pyrazin-3-yl)-1,2,2-trimethylcyclopentanecarboxylate (4 g, 9.98 mmol) and NH₃.H₂O (20 mL) in 2-Propanol (20 mL) was stirred at 110° C. for overnight on a sealed tube. The reaction mixture was concentrated to remove solvent. And then the mixture was added water (50 mL), extracted with EtOAc, The organic layer was purified with silica gel to give the title compound. ¹H NMR (400 MHz, CDCl₃): δ 7.27-7.26 (d, J=4 Hz, 1H), 7.01-7.00 (m, 1H), 5.73 (br. s., 2H), 3.69 (s, 3H), 3.49 (t, J=9.8 Hz, 1H), 2.80 (dt, J=6.3, 12.7 Hz, 1H), 2.65-2.54 (m, 1H), 2.11-1.99 (m, 1H), 1.64 (ddd, J=4.3, 9.7, 13.8 Hz, 1H), 1.36 (s, 3H), 1.12 (s, 3H), 0.75 (s, 3H) ppm.

### Intermediate 15

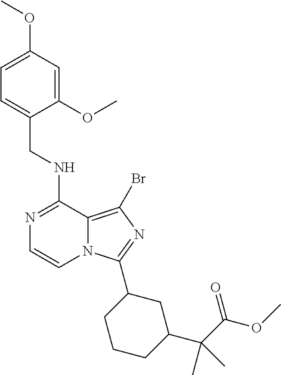

### methyl 2-(3-(1-bromo-8-((2,4-dimethoxybenzyl)amino)imidazo[1,5-a]pyrazin-3-yl)cyclohexyl)-2-methylpropanoate

### Step 1: 2-(3-(((3-chloropyrazin-2-yl)methyl)carbamoyl)cyclohexyl)-2-methylpropanoate

To a solution of 3-(1-methoxy-2-methyl-1-oxopropan-2-yl)cyclohexanecarboxylic acid (1500 mg, 6.57 mmol) in THF (20 ml) was added HATU (3748 mg, 9.86 mmol), DIEA (3.44 ml, 19.71 mmol) and (3-chloropyrazin-2-yl)methanamine hydrochloride (1538 mg, 8.54 mmol) in one portion. After the addition was completed, the mixture was stirred at 25° C. for 12 h. The reaction was quenched by addition of water. The mixture was extracted with ethyl acetate several times. The combined organics was washed with brine, dried over anhydrous sodium sulfate, filtered and concentrated in vacuum. The residue was purified by column chromography on silica gel (PE/THF=4) to afford methyl 2-(3-(((3-chloropyrazin-2-yl)methyl)carbamoyl)cyclohexyl)-2-methylpropanoate as a light solid. ¹H NMR (400 MHz, METHANOL-d₄) δ ppm 1.01-1.07 (m, 1H) 1.12 (s, 6H) 1.26 (d, J=12.13 Hz, 1H) 1.32-1.38 (m, 2H) 1.57-1.62 (m, 1H) 1.69-1.77 (m, 2H) 1.86 (br. s., 2H) 2.30-2.36 (m, 1H) 3.65 (s, 3H) 4.60 (s, 2H) 8.31-8.34 (m, 1H) 8.50-8.52 (m, 1H) ppm.

### Step 2: methyl 2-(3-(8-chloroimidazo[1,5-a]pyrazin-3-yl)cyclohexyl)-2-methylpropanoate

To a solution of methyl 2-(3-(((3-chloropyrazin-2-yl)methyl)carbamoyl)cyclohexyl)-2-methylpropanoate (4000 mg, 11.30 mmol) in MeCN (35 ml) was added PCl₅ (1.18E+04 mg, 56.5 mmol) in one portion. After the addition was completed, the mixture was stirred at 75° C. for 2 h. After cooled to room temperature, the mixture was poured to the solution of NaOH and keep the PH>10. The mixture was extracted with ethyl acetate several times. The combined organic extracts was washed with brine, dried over anhydrous sodium sulfate, filtered and concentrated in vacuum. The residue was purified by column chromatography on silica gel (PE:THF=3:1) to afford methyl 2-(3-(8-chloroimidazo[1,5-a]pyrazin-3-yl)cyclohexyl)-2-methylpropanoate as a light solid. ¹H NMR (400 MHz, METHANOL-d₄) δ ppm 1.15 (s, 6H) 1.56 (d, J=12.13 Hz, 3H) 1.68-1.72 (m, 1H) 1.82-1.87 (m, 1H) 1.89 (s, 2H) 1.90-1.94 (m, 1H) 1.95-1.99 (m, 2H) 3.65 (s, 3H) 7.35 (d, J=5.09 Hz, 1H) 7.80 (s, 1H) 8.16 (d, J=5.09 Hz, 1H) ppm.

### Step 3: methyl 2-(3-(1-bromo-8-chloroimidazo[1,5-a]pyrazin-3-yl)cyclohexyl)-2-methylpropanoate

To a solution of methyl 2-(3-(8-chloroimidazo[1,5-a]pyrazin-3-yl)cyclohexyl)-2-methylpropanoate (2500 mg, 7.44 mmol) in DMF (20 ml) was added NBS (1722 mg, 9.68 mmol) at 0° C. After the addition was completed, the mixture was stirred at this temperature for further half an hour. The reaction was quenched by addition of 15% sodium sulfite. The mixture was extracted with ethyl acetate several times. The combined organics was washed with brine, dried over anhydrous sodium sulfate, filtered and concentrated in vacuum to remove the solvent. The residue was purified by column chromatography on silica gel (PE/THF=3) to afford the methyl 2-(3-(1-bromo-8-chloroimidazo[1,5-a]pyrazin-3-yl)cyclohexyl)-2-methylpropanoate as a light solid. ¹H NMR (400 MHz, METHANOL-d₄) δ ppm 1.16 (d, J=1.96 Hz, 6H) 1.18-1.24 (m, 1H) 1.49-1.58 (m, 3H) 1.67-1.73 (m, 1H) 2.01 (s, 5H) 3.65 (d, J=1.17 Hz, 3H) 7.22-7.34 (m, 1H) 7.85-8.08 (m, 1H) ppm.

Step 4: methyl 2-(3-(1-bromo-8-((2,4-dimethoxybenzyl)amino)imidazo[1,5-a]pyrazin-3-yl)cyclohexyl)-2-methylpropanoate

To a solution of methyl 2-(3-(8-amino-1-bromoimidazo[1,5-a]pyrazin-3-yl)cyclohexyl)-2-methylpropanoate (2600 mg, 6.58 mmol) in DMF (20 ml) was added K₂CO₃ (2727 mg, 19.73 mmol) and (2,4-dimethoxyphenyl)methanamine (3299 mg, 19.73 mmol) in one portion. After the addition was completed, the mixture was stirred at 100° C. for 12 h. After cooled to room temperature, water was added. The mixture was extracted with ethyl acetate several times. The combined organics was washed with brine, dried over anhydrous sodium sulfate, filtered and concentrated in vacuum. The residue was purified by column chromography on silica gel (PE/THF=4) to afford methyl 2-(3-(1-bromo-8-((2,4-dimethoxybenzyl)amino)imidazo[1,5-a]pyrazin-3-yl)cyclohexyl)-2-methylpropanoate as a light oil. ¹H NMR (400 MHz, CHLOROFORM-d) δ ppm 1.04-1.09 (m, 1H) 1.11 (d, J=3.91 Hz, 6H) 1.39-1.51 (m, 2H) 1.52-1.68 (m, 3H) 1.74-1.84 (m, 2H) 1.91 (br. s., 2H) 3.63 (s, 3H) 3.78 (s, 3H) 3.86 (s, 3H) 4.65 (d, J=5.48 Hz, 2H) 6.40-6.44 (m, 1H) 6.47 (s, 1H) 6.99-7.03 (m, 1H) 7.05 (s, 1H) 7.24 (t, J=3.91 Hz, 1H) ppm.

### EXAMPLES

### Example 1

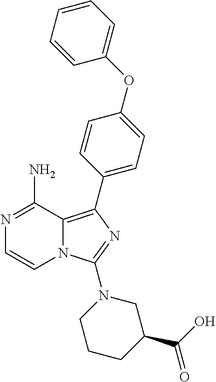 [SMILES: Nc1nccn2c(N3CCC[C@H](C(=O)O)C3)nc(-c3ccc(Oc4ccccc4)cc3)c12]

### (S)-1-(8-amino-1-(4-phenoxyphenyl)imidazo[1,5-a]pyrazin-3-yl)piperidine-3-carboxylic acid

A solution of (S)-methyl 1-(8-amino-1-bromoimidazo[1,5-a]pyrazin-3-yl)piperidine-3-carboxylate (80 mg, 0.15 mmol) and 4,4,5,5-tetramethyl-2-(4-phenoxyphenyl)-1,3,2-dioxaborolane (65.5 mg, 0.3 mmol) in dioxane (1.5 mL) was added a solution of K₂CO₃ (95.2 mg, 0.6 mmol), pd (dppf)Cl₂ (10.35 mg, 0.014 mmol), then the mixture was heated at 100° C. for 1 hour. After the reaction, the mixture was cooled to room temperature and purified by pre-HPLC to give (S)-1-(8-amino-1-(4-phenoxyphenyl)imidazo[1,5-a]pyrazin-3-yl)piperidine-3-carboxylic acid. 1HNMR (400 MHz, CDCl₃): δ=13.79 (br. s., 1H), 11.22 (br. s., 1H), 8.26-8.15 (m, 1H), 7.56 (d, J=8.5 Hz, 2H), 7.41 (t, J=7.5 Hz, 2H), 7.27 (s, 1H), 7.21 (d, J=7.5 Hz, 1H), 7.11 (dd, J=8.0, 16.1 Hz, 4H), 6.11 (br. s., 1H), 4.00-3.88 (m, 1H), 3.57 (d, J=11.5 Hz, 1H), 3.24 (br. s., 1H), 3.01-2.93 (m, 1H), 2.85 (br. s., 1H), 2.48 (d, J=11.5 Hz, 1H), 1.77 (br. s., 1H), 1.37-1.19 (m, 1H) ppm. MS (ESI): M/Z (M+1)⁺=429.5.

The following examples in Table 1 were prepared in the same procedure as example 1, using different intermediates with aryl bromo to couple with biarylether boronic acids for the Suzuki reactions.

TABLE 1

| Example | | | Exact Mass | Retention time |
|---|---|---|---|---|
| Number | Structure | IUPAC Name | [M + H]⁺ | (min, Method) |
| 2 | 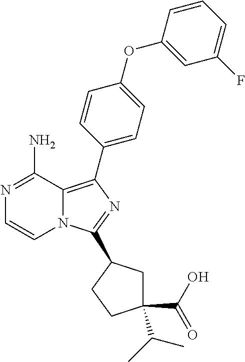 [SMILES: CC(C)[C@]1(C(=O)O)CC[C@@H](c2nc(-c3ccc(Oc4cccc(F)c4)cc3)c3c(N)nccn23)C1] | (1S,3R)-3-{8-amino-1- [4-(3- fluorophenoxy)phenyl] imidazo[1,5-a]pyrazin- 3-yl}-1-(1- methylethyl)cyclo- pentanecarboxylic acid | 475.2 | 1.12 (D) |
| 3 | 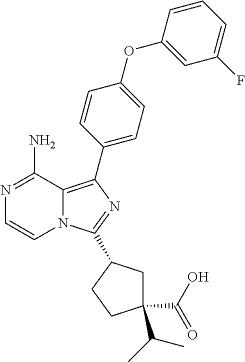 [SMILES: CC(C)[C@@]1(C(=O)O)CC[C@H](c2nc(-c3ccc(Oc4cccc(F)c4)cc3)c3c(N)nccn23)C1] | (1R,3S)-3-{8-amino-1- [4-(3- fluorophenoxy)phenyl] imidazo[1,5-a]pyrazin- 3-yl}-1-(1- methylethyl)cyclo- pentanecarboxylic acid | 475.2 | 1.02 (D) |
| 4 | 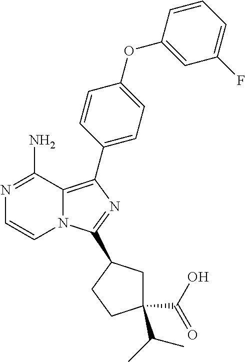 [SMILES: CC(C)[C@@]1(C(=O)O)CC[C@@H](c2nc(-c3ccc(Oc4cccc(F)c4)cc3)c3c(N)nccn23)C1] | (1R,3S)-3-{8-amino-1- [4-(3- fluorophenoxy)phenyl] imidazo[1,5-a]pyrazin- 3-yl}-1-(1- methylethyl)cyclo- pentanecarboxylic acid | 475.2 | 1.2 (D) |
| 5 | 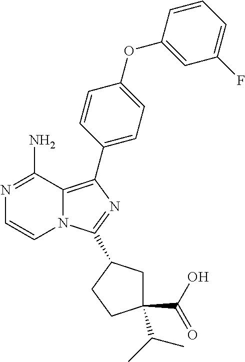 [SMILES: CC(C)[C@]1(C(=O)O)CC[C@H](c2nc(-c3ccc(Oc4cccc(F)c4)cc3)c3c(N)nccn23)C1] | (1R,3S)-3-{8-amino-1- [4-(3- fluorophenoxy)phenyl] imidazo[1,5-a]pyrazin- 3-yl}-1-(1- methylethyl)cyclo- pentanecarboxylic acid | 475.2 | 0.98 (D) |
| 6 | 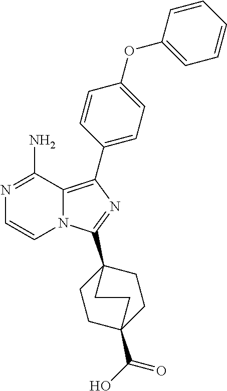 [SMILES: Nc1nccn2c1c(-c1ccc(Oc3ccccc3)cc1)nc2[C@]12CC[C@](C(=O)O)(CC1)CC2] | 4-[8-amino-1-(4- phenoxyphenyl) imidazo[1,5-a]pyrazin-3- yl]bicyclo[2.2.2]octane- 1-carboxylic acid | 455.2 | 0.96 (B) |
| 7 | 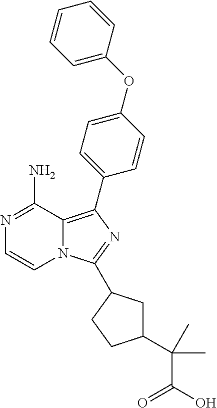 [SMILES: CC(C)(C(=O)O)C1CCC(c2nc(-c3ccc(Oc4ccccc4)cc3)c3c(N)nccn23)C1] | 2-{3-[8-amino-1-(4- phenoxyphenyl) imidazo[1,5-a]pyrazin-3- yl]cyclopentyl}-2- methylpropanoic acid | 457.2 | 1.93 (C) |
| 8 | 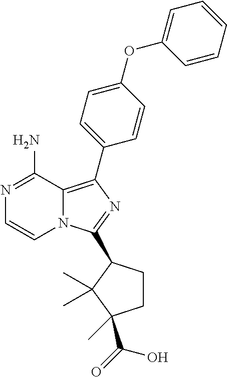 [SMILES: CC1(C)[C@@H](c2nc(-c3ccc(Oc4ccccc4)cc3)c3c(N)nccn23)CC[C@@]1(C)C(=O)O] | (1R,3S)-3-[8-amino-1- (4- phenoxyphenyl)imidazo [1,5-a]pyrazin-3-yl]- 1,2,2- trimethylcyclopenta- carboxylic acid | 457.2 | 1.85 (C) |
| 9 | 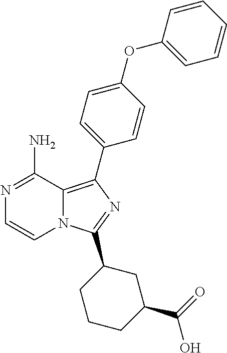 [SMILES: Nc1nccn2c([C@@H]3CCC[C@H](C(=O)O)C3)nc(-c3ccc(Oc4ccccc4)cc3)c12] | (1S,3R)-3-[8-amino-1- (4- phenoxyphenyl) imidazo[1,5-a]pyrazin-3- yl]cyclohexanecarboxylic acid | 429.1 | 2.69 (A) |
| 10 | 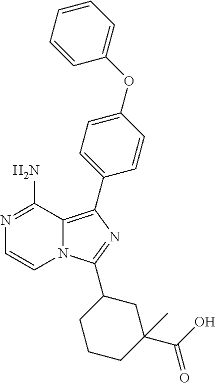 [SMILES: CC1(C(=O)O)CCCC(c2nc(-c3ccc(Oc4ccccc4)cc3)c3c(N)nccn23)C1] | 3-[8-amino-1-(4- phenoxyphenyl)imidazo [1,5-a]pyrazin-3-yl]- 1- mehtylcyclohexane- carboxylic acid | 443.1 | 1.39 (D) |
| 11 | 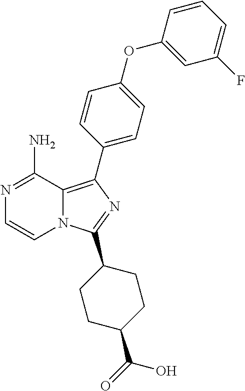 [SMILES: Nc1nccn2c1c(-c1ccc(Oc3cccc(F)c3)cc1)nc2[C@H]1CC[C@@H](C(=O)O)CC1] | cis-4-{8-amino-1-[4- (3- fluorophenoxy)phenyl] imidazo[1,5-a]pyrazin- 3- yl}cyclohexanecarboxylic acid | 447.2 | 1.2 (D) |
| 12 | 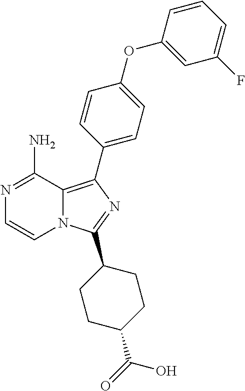 [SMILES: Nc1nccn2c1c(-c1ccc(Oc3cccc(F)c3)cc1)nc2[C@H]1CC[C@H](C(=O)O)CC1] | trans-4-{8-amino-1-[4- (3- fluorophenoxy)phenyl] imidazo[1,5-a]pyrazin- 3- yl}cyclohexanecarboxylic acid | 447.1 | 1.1 (D) |
| 13 | 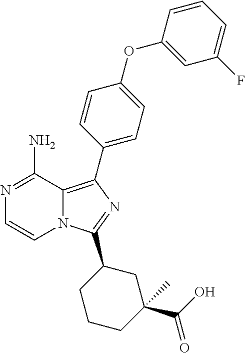 [SMILES: C[C@]1(C(=O)O)CCC[C@@H](c2nc(-c3ccc(Oc4cccc(F)c4)cc3)c3c(N)nccn23)C1] | (1S,3R)-3-{8-amino-1- [4-(3- fluorophenoxy)phenyl] imidazo[1,5-a]pyrazin- 3-yl}-1- methylcyclohexanecarboxylic acid | 461.1 | 1.29 (D) |
| 14 | 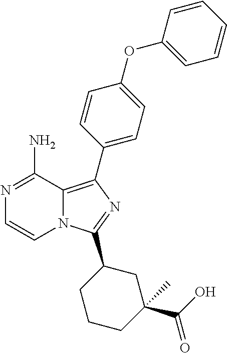 [SMILES: C[C@]1(C(=O)O)CCC[C@@H](c2nc(-c3ccc(Oc4ccccc4)cc3)c3c(N)nccn23)C1] | (1S,3R)-3-[8-amino-1- (4- phenoxyphenyl)imidazo [1,5-a]pyrazin-3-yl]- 1- methylcyclohexane- carboxylic acid | 443.2 | 1.28 (D) |
| 15 | 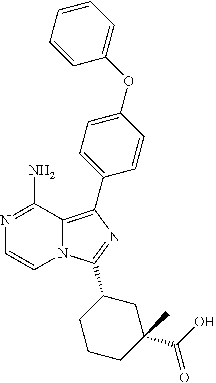 [SMILES: C[C@@]1(C(=O)O)CCC[C@H](c2nc(-c3ccc(Oc4ccccc4)cc3)c3c(N)nccn23)C1] | (1R,3S)-3-[8-amino-1- (4- phenoxyphenyl)imidazo [1,5-a]pyrazin-3-yl]- 1- methylcyclohexane- carboxylic acid | 443.1 | 1.28 (B) |
| 16 | 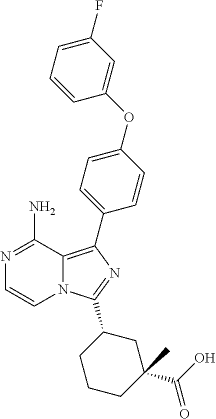 [SMILES: C[C@@]1(C(=O)O)CCC[C@H](c2nc(-c3ccc(Oc4cccc(F)c4)cc3)c3c(N)nccn23)C1] | (1R,3S)-3-{8-amino-1- [4-(3- fluorophenoxy)phenyl] imidazo[1,5-a]pyrazin- 3-yl}-1- methylcyclohexane- carboxylic acid | 461.2 | 1.28 (B) |
| 17 | 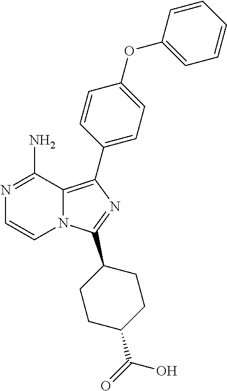 [SMILES: Nc1nccn2c1c(-c1ccc(Oc3ccccc3)cc1)nc2[C@H]1CC[C@H](C(=O)O)CC1] | trans-4-[8-amino-1-(4- phenoxyphenyl)imidazo [1,5-a]pyrazin-3- yl]cyclohexane- carboxylic acid | 429.1 | 1.22 (D) |

### Example 18

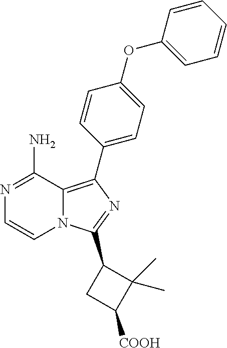 [SMILES: CC1(C)[C@@H](C(=O)O)C[C@H]1c1nc(-c2ccc(Oc3ccccc3)cc2)c2c(N)nccn12]

### (1 S,3R)-3-(8-amino-1-(4-phenoxyphenyl)imidazo[1,5-a]pyrazin-3-1)-2,2-dimethylcyclobutanecarboxylic acid

### Step 1: (1S,3R)-methyl 3-(((3-chloropyrazin-2-yl)(4-phenoxyphenyl)methyl)carbamoyl)-22-dimethylcyclobutanecarboxylate

To a solution of (1R,3S)-3-(methoxycarbonyl)-2,2-dimethylcyclobutanecarboxylic acid (107 mg, 0.576 mmol) and TEA (174 mg, 1.73 mmol) in anhydrous THF (10 mL) was added T₃P (403 mg, 1.27 mmol) at 0° C. The mixture was stirred at this temperature for 30 min. (3-chloropyrazin-2-yl)(4-phenoxyphenyl)methanamine hydrochloride (200 mg, 0.576 mmol) was added in aboved solution. The mixture was stirred at 25° C. for further 2 hours. The mixture was treated with ethyl acetate and water. The ethyl acetate layer was separated and was washed with brine, dried over Na₂SO₄, filtered and concentrated. The residue was purified by silica gel column chromatography (PE/THF=3/1) to afford (1S,3R)-methyl 3-(((3-chloropyrazin-2-yl)(4-phenoxyphenyl)methyl)carbamoyl)-2,2-dimethylcyclobutanecarboxylate as a solid. MS-ESI (m/z): 480.2 (M+1)⁺ (Method D; Rt: 1.31 min).

### Step 2: (1S,3R)-methyl 3-(8-chloro-1-(4-phenoxyphenyl)imidazo[1,5-a]pyrazin-3-yl)-2,2-dimethyl cyclobutanecarboxylate

To a mixture of (1S,3R)-methyl 3-(((3-chloropyrazin-2-yl)(4-phenoxyphenyl)methyl)carbamoyl)-2,2-dimethylcyclobutanecarboxylate (120 mg, 0.26 mmol) in MeCN (10 mL) was added PCl₅ (258 mg, 1.3 mmol), and the mixture was stirred at 60° C. for 2 hours. The reaction was quenched with saturated NaHCO₃ solution and extracted with EA. The EA layer was washed with brine and dried over anhydrous Na₂SO₄. Filtered and the filtrate was concentrated. The residue was purified by silica gel column chromatography (PE/THF=3/1) to afford (1S,3R)-methyl 3-(8-chloro-1-(4-phenoxyphenyl)imidazo[1,5-a]pyrazin-3-yl)-2,2-di methylcyclobutanecarboxylate as a solid. MS-ESI (m/z): 462.2 (M+1)⁺ (LC-MS Method D Rt: 1.473 min).

### Step 3: (1S,3R)-methyl 3-(8-amino-1-(4-phenoxyphenyl)imidazo[1,5-a]pyrazin-3-yl)-2,2-dimethyl cyclobutanecarboxylate

A solution of (1S,3R)-methyl 3-(8-chloro-1-(4-phenoxyphenyl)imidazo[1,5-a]pyrazin-3-yl)-2,2-dimethylcyclobutanecarboxylate (80 mg, 0.17 mol) in i-PrOH (10 mL) saturated with NH₃ was stirred at 120° C. for 24 hours in a 30 mL of sealed tube. The mixture was concentrated to give (1S,3R)-methyl 3-(8-amino-1-(4-phenoxyphenyl)imidazo[1,5-a]pyrazin-3-yl)-2,2-dimethyl cyclobutanecarboxylate as a solid, which was used in the next step directly. MS-ESI (m/z): 443.2 (M+1)⁺ (LC-MS Method D; Rt: 1.075 min).

### Step 4: (1S,3R)-3-(8-amino-1-(4-phenoxyphenyl)imidazo[1,5-a]pyrazin-3-yl)-2,2-dimethylcyclobutanecarboxylic acid

A mixture of (1S,3R)-methyl 3-(8-amino-1-(4-phenoxyphenyl)imidazo[1,5-a]pyrazin-3-yl)-2,2-dimethylcyclobutanecarboxylate (78 mg, 0.18 mmol) and LiOH (37 mg, 0.88 mmol) in MeOH/THF/H₂O (5 mL/5 mL/2 mL) was stirred at 15° C. for 3 hours. The mixture was adjusted to 6 with 1 N HCl. The mixture was extracted with EA. The EA layer was washed with brine and dried over anhydrous Na₂SO₄. Filtered and the filtrate was concentrated. The residue was purified by pre-HPLC to give (1S,3R)-3-(8-amino-1-(4-phenoxyphenyl)imidazo[1,5-a]pyrazin-3-yl)-2,2-dimethylcyclobutanecarboxylic acid. ¹H NMR (CD₃OD, 400 MHz) δ 7.65-7.70 (m, 2H), 7.63 (d, J=6.02 Hz, 1H), 7.38-7.45 (m, 2H), 7.09-7.22 (m, 5H), 6.97 (d, J=6.02 Hz, 1H), 3.75 (dd, J=10.16, 8.16 Hz, 1H), 3.16 (q, J=10.62 Hz, 1H), 3.03-3.09 (m, 1H), 2.31 (dt, J=11.04, 7.91 Hz, 1H), 1.51 (s, 3H), 0.84 (s, 3H) ppm.

The following examples in Table 2 were prepared in the same procedure as example 18, using corresponding diacid monoesters for step 1 in example 18.

TABLE 2

| | | | | Retention |
|---|---|---|---|---|
| Example | | | Exact Mass | time (min, |
| Number | Structure | IUPAC Name | [M + H]⁺ | Method) |
| 19 | 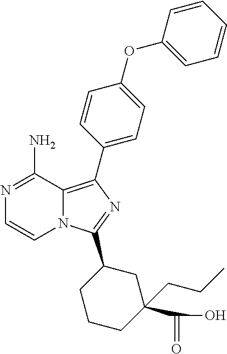 [SMILES: CCC[C@]1(C(=O)O)CCC[C@@H](c2nc(-c3ccc(Oc4ccccc4)cc3)c3c(N)nccn23)C1] | (1S,3R)-3-[8- amino-1-(4- pheenoxyphenyl) imidazo[1,5-a]pyrazin- 3-yl]-1- propylcyclohexane- carboxylic acid | 471.2 | 1.13 (D) |
| 20 | 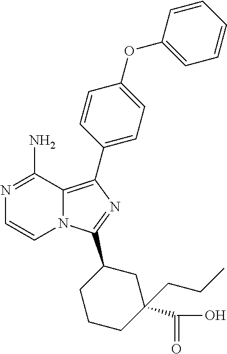 [SMILES: CCC[C@@]1(C(=O)O)CCC[C@@H](c2nc(-c3ccc(Oc4ccccc4)cc3)c3c(N)nccn23)C1] | (1R,3R)-3-[8- amino-1-(4- phenoxyphenyl) imidazo[1,5-a]pyrazin- 3-yl]-1- propylcyclohexane- carboxylic acid | 471.2 | 1.13 (D) |
| 21 | 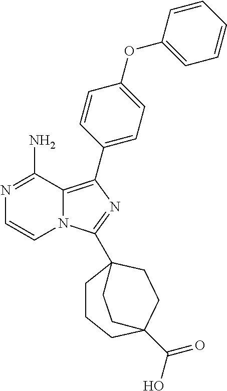 [SMILES: Nc1nccn2c(C34CCCC(C(=O)O)(CC3)CC4)nc(-c3ccc(Oc4ccccc4)cc3)c12] | 5-[8-amino-1-(4- phenoxyphenyl) imidazo[1,5-a]pyrazin- 3- yl]bicyclo[3.2.2] nonane-1-carboxylic acid | 469.2 | 1.07 (D) |
| 22 | 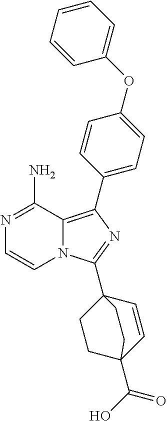 [SMILES: Nc1nccn2c(C34C=CC(C(=O)O)(CC3)CC4)nc(-c3ccc(Oc4ccccc4)cc3)c12] | 4-[8-amino-1-(4- phenoxyphenyl) imidazo[1,5-a]pyrazin- 3- yl]bicyclo[2.2.2]oct- 2-ene-1-carboxylic acid | 453.2 | 2.21 (C) |
| 23 | 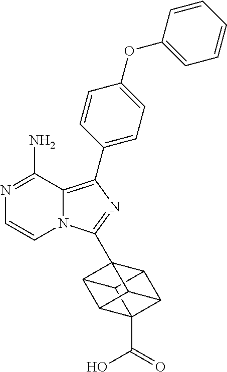 [SMILES: Nc1nccn2c(C34C5C6C3C3C4C5C63C(=O)O)nc(-c3ccc(Oc4ccccc4)cc3)c12] | 4-(8-amino-1-(4- phenoxyphenyl) imidazo[1,5-a]pyrazin- 3-yl)cubane-1- carboxylic acid | 449.2 | 0.91 (D) |
| 24 | 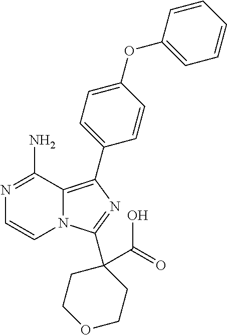 [SMILES: Nc1nccn2c(C3(C(=O)O)CCOCC3)nc(-c3ccc(Oc4ccccc4)cc3)c12] | 4-[8-amino-1-(4- phenoxyphenyl) imidazo[1,5-a]pyrazin- 3-yl]tetrahydro-2H- pyran-4-carboxylic acid | 431.2 | 0.95 (D) |
| 25 | 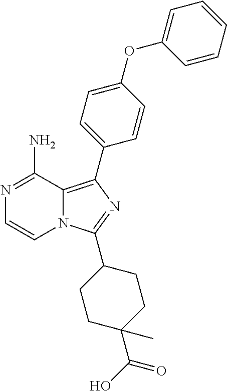 [SMILES: CC1(C(=O)O)CCC(c2nc(-c3ccc(Oc4ccccc4)cc3)c3c(N)nccn23)CC1] | Trans-4-[8-amino- 1-(4- phenoxyphenyl) imidazo[1,5-a]pyrazin- 3-yl]-1- methylcyclohexane carboxylic acid | 443.2 | 1.19 (D) |
| 26 | 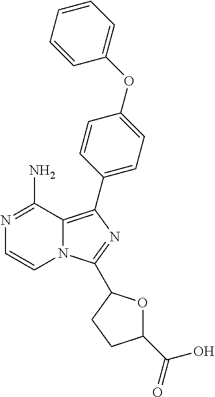 [SMILES: Nc1nccn2c(C3CCC(C(=O)O)O3)nc(-c3ccc(Oc4ccccc4)cc3)c12] | 5-[8-amino-1-(4- phenoxyphenyl) imidazo[1,5-a]pyrazin- 3- yl]tetrahydrofuran- 2-carboxylic acid (E1) | 417.2 | 0.93 (D) |
| 27 | 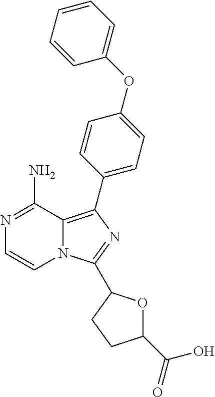 [SMILES: Nc1nccn2c(C3CCC(C(=O)O)O3)nc(-c3ccc(Oc4ccccc4)cc3)c12] | 5-[8-amino-1-(4- phenoxyphenyl) imidazo[1,5-a]pyrazin- 3- yl]tetrahydrofuran- 2-carboxylic acid (E2) | 417.2 | 0.91 (D) |
| 28 | 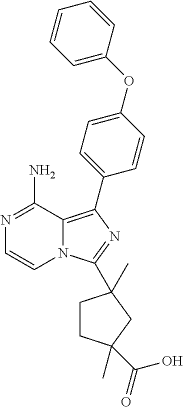 [SMILES: CC1(C(=O)O)CCC(C)(c2nc(-c3ccc(Oc4ccccc4)cc3)c3c(N)nccn23)C1] | 3-[8-amino-1-(4- phenoxyphenyl) imidazo[1,5-a]pyrazin- 3-yl]-1,3- dimethylcyclo- pentanecarboxylic acid (E1) | 443.2 | 1.98 (C) |
| 29 | 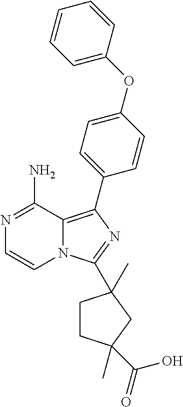 [SMILES: CC1(C(=O)O)CCC(C)(c2nc(-c3ccc(Oc4ccccc4)cc3)c3c(N)nccn23)C1] | 3-[8-amino-1-(4- phenoxyphenyl) imidazo[1,5-a]pyrazin- 3-yl]-1,3- dimethylcyclo- pentanecarboxylic acid (E2) | 443.2 | 1.86 (C) |

### Example 30

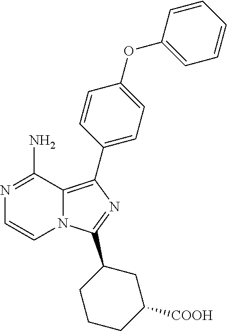 [SMILES: Nc1nccn2c([C@@H]3CCC[C@@H](C(=O)O)C3)nc(-c3ccc(Oc4ccccc4)cc3)c12]

### Trans-3-(8-amino-1-(4-phenoxyphenyl)imidazo[1,5-a]pyrazin-3-yl)cyclohexanecarboxylic acid

### Step 1: trans-3-(((3-chloropyrazin-2-yl)(4-phenoxyphenyl)methyl)carbamoyl)cyclohexanecarboxylic acid

To a solution of trans-cyclohexane-1,3-dicarboxylic acid (195 mg, 1.132 mmol) and TEA (238 mg, 2.4 mmol) in anhydrous THF (10 mL) was added HATU (430 mg, 1.1 mmol) at 0° C. The mixture was stirred at this temperature for 30 min. (3-chloropyrazin-2-yl)(4-phenoxy phenyl)methanamine hydrochloride (200 mg, 0.576 mmol) was added in aboved solution, and the resulting mixture was stirred at 25° C. for 2 hours. The mixture was treated with EA and water. The EA layer was separated and was washed with brine, dried over Na₂SO₄, filtered and concentrated. The residue was purified by silica gel column chromatography (PE/THF=3/1) to afford trans-3-(((3-chloropyrazin-2-yl)(4-phenoxyphenyl)methyl)carbamoyl)cyclohexane carboxylic acid as a solid. MS-ESI (m/z): 466.2 (M+1)⁺ (Acq Method D; Rt: 1.247 min).

### Step 2: trans-ethyl 3-(((3-chloropyrazin-2-yl)(4-phenoxyphenyl)methyl)carbamoyl)cyclohexanecarboxylate

To a solution of trans-3-(((3-chloropyrazin-2-yl)(4-phenoxyphenyl)methyl)carbamoyl)cyclo hexanecarboxylic acid (300 mg, 0.65 mmol) and K₂CO₃ (223 mg, 1.6 mmol) in DMF (5 mL) was added EtI (121 mg, 0.77 mmol). The mixture was stirred at 25° C. for 2 hours. The reaction was treated with EA and water. The EA layer was separated and was washed with brine, dried, filtered and concentrated. The residue was purified by silica gel column chromatography (PE/THF=5/1) to afford trans-ethyl 3-(((3-chloropyrazin-2-yl)(4-phenoxyphenyl)methyl) carbamoyl)cyclohexanecarboxylate as a solid. MS-ESI (m/z): 494.2 (M+1)⁺ (Acq Method D; Rt: 1.367 min).

### Step 3: trans-ethyl 3-(8-chloro-1-(4-phenoxyphenyl)imidazo[1,5-a]pyrazin-3-yl)cyclohexanecarboxylate

To a solution of trans-ethyl 3-(((3-chloropyrazin-2-yl)(4-phenoxyphenyl)methyl)carbamoyl) cyclohexanecarboxylate (200 mg, 0.41 mmol) in MeCN (10 mL) was added PCl₅ (418 mg, 2.03 mmol), and the reaction mixture was stirred at 60° C. for 2 hours. The mixture was quenched with saturated NaHCO₃ solution and extracted with EA. The EA layer was washed with brine and dried over anhydrous Na₂SO₄, filtered and the filtrate was concentrated. The residue was purified by silica gel column chromatography (PE/THF=3/1) to afford trans-ethyl 3-(8-chloro-1-(4-phenoxyphenyl)imidazo[1,5-a]pyrazin-3-yl)cyclohexanecarboxylate as a solid.

MS-ESI (m/z): 476.2 (M+1)⁺ (Acq Method D; Rt: 1.567 min).

### Step 4: trans-ethyl 3-(8-amino-1-(4-phenoxyphenyl)imidazo[1,5-a]pyrazin-3-yl)cyclohexanecarboxylate

A solution of trans-ethyl 3-(8-chloro-1-(4-phenoxyphenyl)imidazo[1,5-a]pyrazin-3-yl)cyclo hexanecarboxylate (150 mg, 0.32 mol) in i-PrOH (10 mL) saturated with NH₃ was stirred at 120° C. for 24 hours in a 30 mL of sealed tube. The mixture was concentrated and the residue was purified by silica gel column chromatography (PE/THF=1/1) to afford trans-ethyl 3-(8-amino-1-(4-phenoxyphenyl)imidazo[1,5-a]pyrazin-3-yl)cyclohexanecarboxylate as a solid.

MS-ESI (m/z): 457.3 (M+1)⁺ (Acq Method D; Rt: 1.127 min).

### Step 5: trans-3-(8-amino-1-(4-phenoxyphenyl)imidazo[1,5-a]pyrazin-3-yl)cyclohexanecarboxylic acid

A mixture of trans-ethyl 3-(8-amino-1-(4-phenoxyphenyl)imidazo[1,5-a]pyrazin-3-yl)cyclo hexanecarboxylate (100 mg, 0.22 mmol) and LiOH (46 mg, 1.1 mmol) in MeOH/THF/H₂O (5 mL/5 mL/2 mL) was stirred at 15° C. for 3 hours. The mixture was adjusted to 6 with 1 N HCl. The mixture was extracted with EA. The EA layer was washed with brine and dried over anhydrous Na₂SO₄. Filtered and the filtrate was concentrated. The residue was purified by pre-HPLC to give trans-3-(8-amino-1-(4-phenoxyphenyl)imidazo[1,5-a]pyrazin-3-yl)cyclo hexanecarboxylic acid. ¹H NMR (CD₃OD, 400 MHz) δ 7.77 (d, J=6.02 Hz, 1H), 7.68 (d, J=8.53 Hz, 2H), 7.39-7.48 (m, 2H), 7.09-7.25 (m, 5H), 7.01 (d, J=6.02 Hz, 1H), 3.46-3.58 (m, 1H), 2.98 (t, J=4.02 Hz, 1H), 2.34 (d, J=13.55 Hz, 1H), 2.20 (d, J=10.04 Hz, 1H), 1.98-2.08 (m, 1H), 1.83-1.95 (m, 3H), 1.71-1.81 (m, 1H), 1.57-1.69 (m, 1H) ppm.

### Biological Activity

The Btk inhibitor compounds of the invention having Formula I inhibit the Btk kinase activity. All compounds of the invention have an IC50 of 10 μM or lower. In another aspect the invention relates to compounds of Formula I which have an IC50 of less than 100 nM. In yet another aspect the invention relates to compounds of Formula I which have an IC50 of less than 10 nM.

The term IC50 means the concentration of the test compound that is required for 50% inhibition of its maximum effect in vitro.

### Btk Enzyme Activity Assay Methods

BTK enzymatic activity was determined with the LANCE (Lanthanide Chelate Excite) TR-FRET (Time-resolved fluorescence resonance energy transfer) assay. In this assay, the potency (IC₅₀) of each compound was determined from an eleven point (1:3 serial dilution; final compound concentration range in assay from 1 μM to 0.017 nM) titration curve using the following outlined procedure. To each well of a black non-binding surface Corning 384-well microplate (Corning Catalog #3820), 5 nL of compound (2000 fold dilution in final assay volume of 10 μL) was dispensed, followed by the addition of 7.5 μL of 1× kinase buffer (50 mM Hepes 7.5, 10 mM MgCl₂, 0.01% Brij-35, 1 mM EGTA, 0.05% BSA & 1 mM DTT) containing 5.09 pg/μL (66.67 pM) of BTK enzyme (recombinant protein from baculovirus-transfected Sf9 cells: full-length BTK, 6HIS-tag cleaved). Following a 60 minute compound & enzyme incubation, each reaction was initiated by the addition of 2.5 μL 1× kinase buffer containing 8 μM biotinylated “A5” peptide (Biotin-EQEDEPEGDYFEWLE-NH2) (SEQ.ID.NO.: 1), and 100 μM ATP. The final reaction in each well of 10 μL consists of 50 pM hBTK, 2 μM biotin-A5-peptide, and 25 μM ATP. Phosphorylation reactions were allowed to proceed for 120 minutes. Reactions were immediately quenched by the addition of 20 uL of 1× quench buffer (15 mM EDTA, 25 mM Hepes 7.3, and 0.1% Triton X-100) containing detection reagents (0.626 nM of LANCE-Eu-W1024-anti-phosphoTyrosine antibody, PerkinElmer and 86.8 nM of Streptavidin-conjugated Dylight 650, Dyomics/ThermoFisher Scientific). After 60 minutes incubation with detection reagents, reaction plates were read on a PerkinElmer EnVision plate reader using standard TR-FRET protocol. Briefly, excitation of donor molecules (Eu-chelate:anti-phospho-antibody) with a laser light source at 337 nm produces energy that can be transferred to Dylight-650 acceptor molecules if this donor:acceptor pair is within close proximity. Fluorescence intensity at both 665 nm (acceptor) and 615 nm (donor) are measured and a TR-FRET ratio calculated for each well (acceptor intensity/donor intensity). IC₅₀ values were determined by 4 parameter robust fit of TR-FRET ratio values vs. (Log₁₀) compound concentrations.

The following Table 3 provides specific IC50 values for all the examples. The IC50 values set forth below were determined according to Assay method described above.

TABLE 3
BTK binding potency

| Example | BTK binding IC50 |
|---|---|
| number | (nM) |
| Example 1 | 2.7 |
| Example 2 | 2.0 |
| Example 3 | 19.5 |
| Example 4 | 4.4 |
| Example 5 | 1.4 |
| Example 6 | 0.09 |
| Example 7 | 2.1 |
| Example 8 | 0.7 |
| Example 9 | 7.1 |
| Example 10 | 1.6 |
| Example 11 | 14.3 |
| Example 12 | 1.0 |
| Example 13 | 5.9 |
| Example 14 | 2.4 |
| Example 15 | 1.3 |
| Example 16 | 3.3 |
| Example 17 | 0.5 |
| Example 18 | 3.5 |
| Example 19 | 200.4 |
| Example 20 | 4.9 |
| Example 21 | 0.14 |
| Example 22 | 0.26 |
| Example 23 | 0.30 |
| Example 24 | 79.6 |
| Example 25 | 12.3 |
| Example 26 | 12.5 |
| Example 27 | 12.5 |
| Example 28 | 0.23 |
| Example 29 | 1.3 |
| Example 30 | 0.18 |

What is claimed is:

1. A compound according to Formula I, or a pharmaceutically acceptable salt thereof 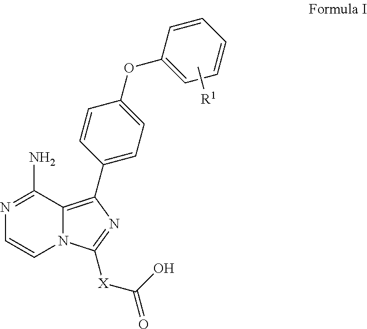
wherein:
R¹ is hydrogen or halogen;
X is selected from the group consisting of:
a) cycloalkyl;
b) cycloalkylmethylene;
c) cycloalkenyl; and
d) heterocycloalkyl;
each optionally substituted with one, two or three (1-6C)alkyl groups.

2. The compound of claim 1, or a pharmaceutically acceptable salt thereof, wherein X is selected from the group consisting of:
a) cyclobutyl;
b) cyclopentyl;
c) cyclohexyl;
d) bicyclo[2.2.2]octanyl;
e) bicyclo[2.2.3]nonanyl;
f) bicyclo[2.2.2]octenyl;
g) cubanyl;
h) tetrahydrofuranyl;
i) tetrahydropyranyl; and
j) piperidinyl.

3. The compound of claim 1, or a pharmaceutically acceptable salt thereof, wherein R¹ is halogen.

4. The compound of claim 1 selected from the group consisting of:
(3R)-1-[8-amino-1-(4-phenoxyphenyl)imidazo[1,5-a]pyrazin-3-yl]piperidine-3-carboxylic acid;
(1 S,3R)-3-{8-amino-1-[4-(3-fluorophenoxy)phenyl]imidazo[1,5-a]pyrazin-3-yl}-1-(1-methylethyl)cyclopentanecarboxylic acid;
(1R,3S)-3-{8-amino-1-[4-(3-fluorophenoxy)phenyl]imidazo[1,5-a]pyrazin-3-yl}-1-(1-methylethyl)cyclopentanecarboxylic acid;
(1R,3R)-3-{8-amino-1-[4-(3-fluorophenoxy)phenyl]imidazo[1,5-a]pyrazin-3-yl}-1-(1-methylethyl)cyclopentanecarboxylic acid;
(1 S,3S)-3-{8-amino-1-[4-(3-fluorophenoxy)phenyl]imidazo[1,5-a]pyrazin-3-yl}-1-(1-methylethyl)cyclopentanecarboxylic acid;
4-[8-amino-1-(4-phenoxyphenyl)imidazo[1,5-a]pyrazin-3-yl]bicyclo[2.2.2]octane-1-carboxylic acid;
2-{3-[8-amino-1-(4-phenoxyphenyl)imidazo[1,5-a]pyrazin-3-yl]cyclopentyl}-2-methylpropanoic acid;
(1R,3S)-3-[8-amino-1-(4-phenoxyphenyl)imidazo[1,5-a]pyrazin-3-yl]-1,2,2-trimethylcyclopentanecarboxylic acid;
3-[8-amino-1-(4-phenoxyphenyl)imidazo[1,5-a]pyrazin-3-yl]-1-methylcyclohexanecarboxylic acid;
(1 S,3R)-3-[8-amino-1-(4-phenoxyphenyl)imidazo[1,5-a]pyrazin-3-yl]cyclohexanecarboxylic acid;
cis-4-{8-amino-1-[4-(3-fluorophenoxy)phenyl]imidazo[1,5-a]pyrazin-3-yl}cyclohexanecarboxylic acid;
trans-4-{8-amino-1-[4-(3-fluorophenoxy)phenyl]imidazo[1,5-a]pyrazin-3-yl}cyclohexanecarboxylic acid;
(1 S,3R)-3-{8-amino-1-[4-(3-fluorophenoxy)phenyl]imidazo[1,5-a]pyrazin-3-yl}-1-methylcyclohexanecarboxylic acid;
(1 S,3R)-3-[8-amino-1-(4-phenoxyphenyl)imidazo[1,5-a]pyrazin-3-yl]-1-methylcyclohexanecarboxylic acid;
(1R,3S)-3-[8-amino-1-(4-phenoxyphenyl)imidazo[1,5-a]pyrazin-3-yl]-1-methylcyclohexanecarboxylic acid;
(1R,3S)-3-{8-amino-1-[4-(3-fluorophenoxy)phenyl]imidazo[1,5-a]pyrazin-3-yl}-1-methylcyclohexanecarboxylic acid;
trans-4-[8-amino-1-(4-phenoxyphenyl)imidazo[1,5-a]pyrazin-3-yl]cyclohexanecarboxylic acid;
(1 S,3R)-3-[8-amino-1-(4-phenoxyphenyl)imidazo[1,5-a]pyrazin-3-yl]-2,2-dimethylcyclobutanecarboxylic acid;
(1 S,3R)-3-[8-amino-1-(4-phenoxyphenyl)imidazo[1,5-a]pyrazin-3-yl]-1-propylcyclohexanecarboxylic acid;
(1R,3R)-3-[8-amino-1-(4-phenoxyphenyl)imidazo[1,5-a]pyrazin-3-yl]-1-propylcyclohexanecarboxylic acid;
5-[8-amino-1-(4-phenoxyphenyl)imidazo[1,5-a]pyrazin-3-yl]bicyclo[3.2.2]nonane-1-carboxylic acid;
4-[8-amino-1-(4-phenoxyphenyl)imidazo[1,5-a]pyrazin-3-yl]bicyclo[2.2.2]oct-2-ene-1-carboxylic acid;
4-(8-amino-1-(4-phenoxyphenyl)imidazo[1,5-a]pyrazin-3-yl)cubane-1-carboxylic acid;
4-[8-amino-1-(4-phenoxyphenyl)imidazo[1,5-a]pyrazin-3-yl]tetrahydro-2H-pyran-4-carboxylic acid;
4-[8-amino-1-(4-phenoxyphenyl)imidazo[1,5-a]pyrazin-3-yl]-1-methylcyclohexanecarboxylic acid;
5-[8-amino-1-(4-phenoxyphenyl)imidazo[1,5-a]pyrazin-3-yl]tetrahydrofuran-2-carboxylic acid;
5-[8-amino-1-(4-phenoxyphenyl)imidazo[1,5-a]pyrazin-3-yl]tetrahydrofuran-2-carboxylic acid;
3-[8-amino-1-(4-phenoxyphenyl)imidazo[1,5-a]pyrazin-3-yl]-1,3-dimethylcyclopentanecarboxylic acid;
3-[8-amino-1-(4-phenoxyphenyl)imidazo[1,5-a]pyrazin-3-yl]-1,3-dimethylcyclopentanecarboxylic acid; and
(1R,3R)-3-[8-amino-1-(4-phenoxyphenyl)imidazo[1,5-a]pyrazin-3-yl]cyclohexanecarboxylic acid;
or a pharmaceutically acceptable salt thereof.

5. A pharmaceutical composition which comprises the compound of claim 1 or a pharmaceutically acceptable salt thereof and one or more pharmaceutically acceptable carriers.

6. The pharmaceutical composition of claim 5, which further comprises at least one additional therapeutically active agent.

7. The compound of claim 1 or a pharmaceutically acceptable salt thereof for use in therapy.

8. The compound of claim 1 or a pharmaceutically acceptable salt thereof for use in the treatment of Bruton's Tyrosine Kinase (Btk) mediated disorders.

9. Use of the compound of Formula I according to claim 1 or a pharmaceutically acceptable salt thereof for the manufacture of a medicament for the treatment of Bruton's Tyrosine Kinase (Btk) mediated disorders.

10. A method for treating a subject suffering with a Bruton's Tyrosine Kinase (Btk) mediated disorder comprising administering to the subject the compound of claim 1 in an amount effective to treat the Btk mediated disorder, thereby treating the subject.

11. The method of claim 10, wherein the Btk mediated disorder is selected from the group consisting of rheumatoid arthritis, psoriatic arthritis, infectious arthritis, progressive chronic arthritis, deforming arthritis, osteoarthritis, traumatic arthritis, gouty arthritis, Reiter's syndrome, polychondritis, acute synovitis and spondylitis, glomerulonephritis (with or without nephrotic syndrome), autoimmune hematologic disorders, hemolytic anemia, aplasic anemia, idiopathic thrombocytopenia, and neutropenia, autoimmune gastritis, and autoimmune inflammatory bowel diseases, ulcerative colitis, Crohn's disease, host versus graft disease, allograft rejection, chronic thyroiditis, Graves' disease, schleroderma, diabetes (type I and type II), active hepatitis (acute and chronic), pancreatitis, primary billiary cirrhosis, myasthenia gravis, multiple sclerosis, systemic lupus erythematosis, psoriasis, atopic dermatitis, contact dermatitis, eczema, skin sunburns, vasculitis (e.g. Behcet's disease) chronic renal insufficiency, Stevens-Johnson syndrome, inflammatory pain, idiopathic sprue, cachexia, sarcoidosis, Guillain-Barré syndrome, uveitis, conjunctivitis, kerato conjunctivitis, otitis media, periodontal disease, pulmonary interstitial fibrosis, asthma, bronchitis, rhinitis, sinusitis, pneumoconiosis, pulmonary insufficiency syndrome, pulmonary emphysema, pulmonary fibrosis, silicosis, chronic inflammatory pulmonary disease, and chronic obstructive pulmonary disease.

12. The method of claim 11, wherein the Btk mediated disorder is rheumatoid arthritis, psoriatic arthritis, or osteoarthritis.
